(19) United States
(12) Patent Application Publication
(10) Pub. No.: US 2012/0220453 A1
(43) Pub. Date: Aug. 30, 2012

(54) PESTICIDAL COMPOSTIONS AND PROCESSES RELATED THERETO

(75) Inventors: Christian T. Lowe, Westfield, IN (US); Tony K. Trullinger, Westfield, IN (US); Ricky Hunter, Westfield, IN (US)

(73) Assignee: DOW AGROSCIENCES LLC, Indianapolis, IN (US)

(21) Appl. No.: 13/404,100

(22) Filed: Feb. 24, 2012

Related U.S. Application Data
(60) Provisional application No. 61/446,621, filed on Feb. 25, 2011.

(51) Int. Cl. A01N 43/56; A01C 1/06; A61K 31/444; A01P 5/00; A61K 31/4439; A61P 33/14; A01P 7/02; A01P 7/04; C07D 401/14; C07D 401/04

(52) U.S. Cl. 504/100; 546/256; 514/333; 546/275.4; 514/341

(57) ABSTRACT

This document discloses pesticidal molecules having the following formula (“Formula One”):

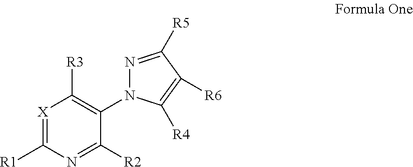

and processes related thereto.

### CROSS-REFERENCE TO RELATED APPLICATIONS

This application claims priority from U.S. provisional application 61/446,621 filed on Feb. 25, 2011. The entire content of this provisional application is hereby incorporated by reference into this Application.

### FIELD OF THE INVENTION

The invention disclosed in this document is related to the field of processes to produce molecules that are useful as pesticides (e.g., acaricides, insecticides, molluscicides, and nematicides), such molecules, and processes of using such molecules to control pests.

### BACKGROUND OF THE INVENTION

Pests cause millions of human deaths around the world each year. Furthermore, there are more than ten thousand species of pests that cause losses in agriculture. The world-wide agricultural losses amount to billions of U.S. dollars each year.

Termites cause damage to all kinds of private and public structures. The world-wide termite damage losses amount to billions of U.S. dollars each year.

Stored food pests eat and adulterate stored food. The world-wide stored food losses amount to billions of U.S. dollars each year, but more importantly, deprive people of needed food.

There is an acute need for new pesticides. Certain pests are developing resistance to pesticides in current use. Hundreds of pest species are resistant to one or more pesticides. The development of resistance to some of the older pesticides, such as DDT, the carbamates, and the organophosphates, is well known. But resistance has even developed to some of the newer pesticides.

Therefore, for many reasons, including the above reasons, a need exists for new pesticides.

### DEFINITIONS

The examples given in the definitions are generally non-exhaustive and must not be construed as limiting the invention disclosed in this document. It is understood that a substituent should comply with chemical bonding rules and steric compatibility constraints in relation to the particular molecule to which it is attached.

“Acaricide Group” is defined under the heading “ACARICIDES”.

“AI Group” is defined after the place in this document where the “Herbicide Group” is defined.

“Alkenyl” means an acyclic, unsaturated (at least one carbon-carbon double bond), branched or unbranched, substituent consisting of carbon and hydrogen, for example, vinyl, allyl, butenyl, pentenyl, and hexenyl.

“Alkenyloxy” means an alkenyl further consisting of a carbon-oxygen single bond, for example, allyloxy, butenyloxy, pentenyloxy, hexenyloxy.

“Alkoxy” means an alkyl further consisting of a carbon-oxygen single bond, for example, methoxy, ethoxy, propoxy, isopropoxy, butoxy, isobutoxy, and tert-butoxy.

“Alkyl” means an acyclic, saturated, branched or unbranched, substituent consisting of carbon and hydrogen, for example, methyl, ethyl, propyl, isopropyl, butyl, and tert-butyl.

“Alkynyl” means an acyclic, unsaturated (at least one carbon-carbon triple bond), branched or unbranched, substituent consisting of carbon and hydrogen, for example, ethynyl, propargyl, butynyl, and pentynyl.

“Alkynyloxy” means an alkynyl further consisting of a carbon-oxygen single bond, for example, pentynyloxy, hexynyloxy, heptynyloxy, and octynyloxy.

“Aryl” means a cyclic, aromatic substituent consisting of hydrogen and carbon, for example, phenyl, naphthyl, and biphenyl.

“Cycloalkenyl” means a monocyclic or polycyclic, unsaturated (at least one carbon-carbon double bond) substituent consisting of carbon and hydrogen, for example, cyclobutenyl, cyclopentenyl, cyclohexenyl, norbornenyl, bicyclo[2.2.2]octenyl, tetrahydronaphthyl, hexahydronaphthyl, and octahydronaphthyl.

“Cycloalkenyloxy” means a cycloalkenyl further consisting of a carbon-oxygen single bond, for example, cyclobutenyloxy, cyclopentenyloxy, norbornenyloxy, and bicyclo[2.2.2]octenyloxy.

“Cycloalkyl” means a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

“Cycloalkoxy” means a cycloalkyl further consisting of a carbon-oxygen single bond, for example, cyclopropyloxy, cyclobutyloxy, cyclopentyloxy, norbornyloxy, and bicyclo[2.2.2]octyloxy.

“Fungicide Group” is defined under the heading “FUNGICIDES.”

“Halo” means fluoro, chloro, bromo, and iodo.

“Haloalkoxy” means an alkoxy further consisting of, from one to the maximum possible number of identical or different, halos, for example, fluoromethoxy, trifluoromethoxy, 2,2-difluoropropoxy, chloromethoxy, trichloromethoxy, 1,1,2,2-tetrafluoroethoxy, and pentafluoroethoxy.

“Haloalkyl” means an alkyl further consisting of, from one to the maximum possible number of, identical or different, halos, for example, fluoromethyl, trifluoromethyl, 2,2-difluoropropyl, chloromethyl, trichloromethyl, and 1,1,2,2-tetrafluoroethyl.

“Herbicide Group” is defined under the heading “HERBICIDES.”

“Heterocyclyl” means a cyclic substituent that may be fully saturated, partially unsaturated, or fully unsaturated, where the cyclic structure contains at least one carbon and at least one heteroatom, where said heteroatom is nitrogen, sulfur, or oxygen. Examples of aromatic heterocyclyls include, but are not limited to, benzofuranyl, benzoisothiazolyl, benzoisoxazolyl, benzoxazolyl, benzothienyl, benzothiazolyl cinnolinyl, furanyl, indazolyl, indolyl, imidazolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, tetrazolyl, thiazolinyl, thiazolyl, thienyl, triazinyl, and triazolyl. Examples of fully saturated heterocyclyls include, but are not limited to, piperazinyl, piperidinyl, morpholinyl, pyrrolidinyl, tetrahydrofuranyl, and tetrahydropyranyl. Examples of partially unsaturated heterocyclyls include, but are not limited to, 1,2,3,4-tetrahydro-quinolinyl, 4,5-dihydro-oxazolyl, 4,5-dihydro-1H-pyrazolyl, 4,5-dihydro-isoxazolyl, and 2,3-dihydro-[1,3,4]-oxadiazolyl.

“Insecticide Group” is defined under the heading “INSECTICIDES.”

“Nematicide Group” is defined under the heading “NEMATICIDES”

“Synergist Group” is defined under the heading “SYNERGISTIC MIXTURES AND SYNERGISTS”

### DETAILED DESCRIPTION OF THE INVENTION

This document discloses molecules having the following formula (“Formula One”):

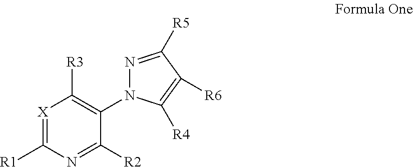

wherein:
(a) X is selected from N or CR12;
(b) R1 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)halo alkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));

(c) R2 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));

(d) R3 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));

(e) R4 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));

(f) R5 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13),

(R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)), or

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13));

(g) R6 is selected from one of the following (6a), (6b), (6c), (6d), (6e), (6f), or (6g)

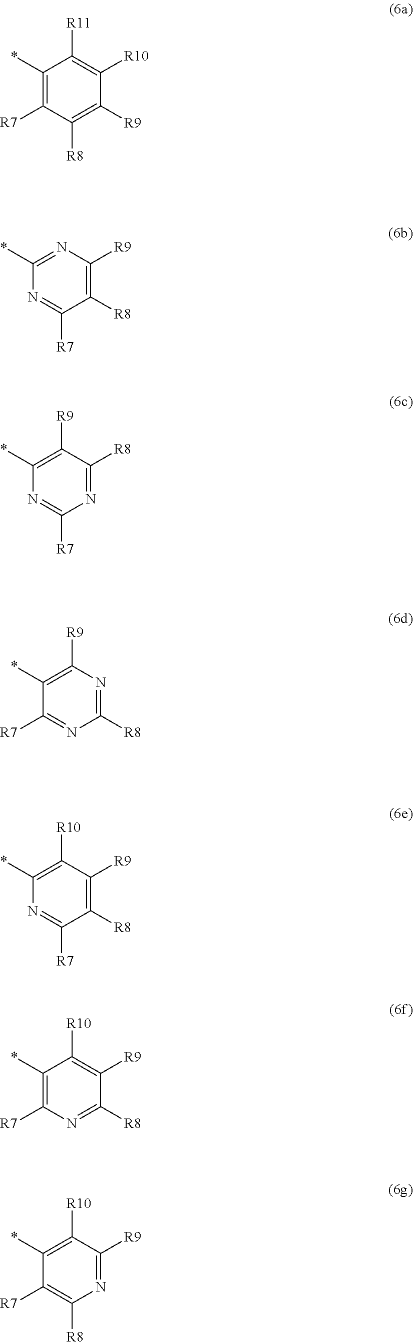

wherein * indicates the bond that is attached to the pyrazolyl ring;

(h) each R7 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(i) each R8 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(j) each R9 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(k) each R10 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(l) each R11 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(m) R12 is selected from

(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));

(n) each R13 is independently selected from

(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,

(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)halo alkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,

(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;

(o) with the following provisos

(1) the following compounds are excluded

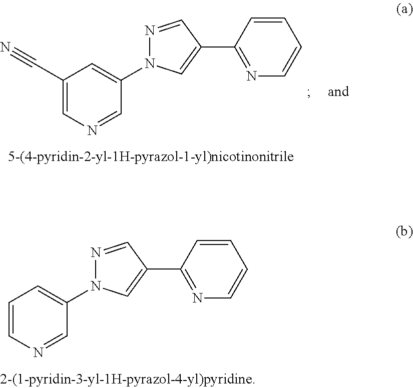

In another embodiment of this invention X is preferably CR12.

In another embodiment of this invention R1 is preferably H.

In another embodiment of this invention R2 is preferably H.

In another embodiment of this invention R3 is preferably H.

In another embodiment of this invention R4 is preferably H.

In another embodiment of this invention R5 is preferably H, (C₁-C₆)alkyl, or halo.

In another embodiment of this invention R5 is preferably CH₃, or Cl.

In another embodiment of this invention R6 is preferably 6(e), 6(f), or 6(g).

In another embodiment of this invention R7 is preferably (C₁-C₆)haloalkyl.

In another embodiment of this invention R8 is preferably (C₁-C₆)haloalkyl.

In another embodiment of this invention R9 is preferably (C₁-C₆)haloalkyl.

In another embodiment of this invention R10 is preferably (C₁-C₆)haloalkyl.

In another embodiment of this invention R11 is preferably (C₁-C₆)haloalkyl.

In another embodiment of this invention R12 is preferably H.

The molecules of Formula One will generally have a molecular mass of about 100 Daltons to about 1200 Daltons. However, it is generally preferred if the molecular mass is from about 120 Daltons to about 900 Daltons, and it is even more generally preferred if the molecular mass is from about 140 Daltons to about 600 Daltons.

Preparation of pyridylpyrazoles such as those of Formula VIa is demonstrated in Scheme I. The compound of Formula IV in step a and as in Cristau, Henri-Jean et al. Eur. J. Org. Chem. 2004, 695-709 can be prepared through the N-arylation of a pyrazole of Formula III with aryl halide, where W is bromine or iodine, of Formula II in the presence of a base such as cesium carbonate, a copper catalyst such as copper (II) oxide and a ligand such as salicylaldoxime in a polar aprotic solvent such as acetonitrile. Alternatively, the compound of Formula IV in step b can be obtained via aryl nucleophilic substitution of Formula II, where W is fluorine, in the presence of the pyrazole of Formula III with a base such as sodium hydride in a polar aprotic solvent such as DMF or DMSO. The preparation of compounds of Formula Va, where Y is bromine, can be achieved as in step c by using a bromine source such as N-bromosuccinimide in a solvent such as acetonitrile. The preparation of compounds of formula Va, where Y is iodine, can be achieved as in step d as in Potapov et al, Russian Journal of Organic Chemistry 2006, 42, 1368-1373 by using an iodine source, such as iodine under acidic conditions such as a mixture of sulfuric acid, acetic acid and iodic acid. The preparation of compounds of Formula VIa, where B(O)₂R14 is a boronic acid or boronic ester, can be achieved as in WO 2008095944 and as in step e using borylation reagents such as bis(pinacolato)diboron with a base, such as potassium acetate, and a catalyst, such as 1,1′-bis(diphenylphosphino)ferrocenepalladium(II) dichloride, in a solvent, such as acetonitrile.

The preparation of compounds of Formula VIa, where B(O)₂R14 is a boronic acid or boronic ester, can be achieved as in Lin, Qiyan et al. Organic Letters (2009), 11(9), 1999-2002 and as in step f via halogen-magnesium exchange of compounds of Formula Va with isopropylmagnesium chloride followed by addition borate such as 2-isopropoxy-4,4,5,5-tetramethyl-1,3,2-dioxaborolane or triisopropyl borate in a polar aprotic solvent such as THF.

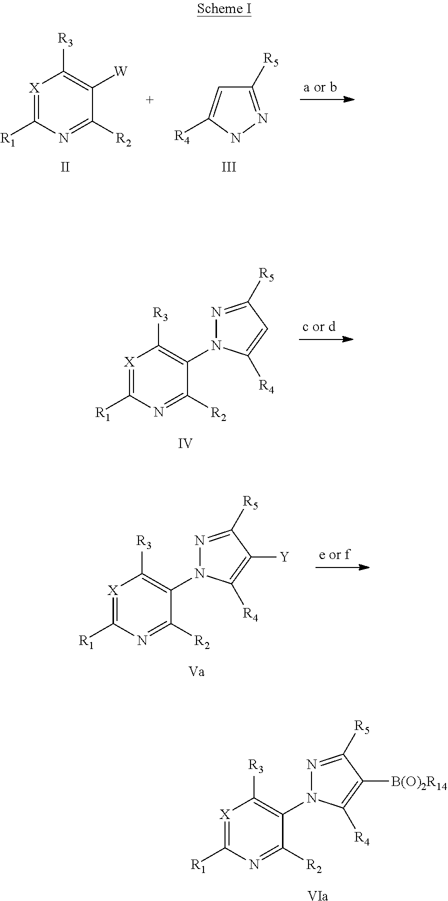

Compounds of Formula Ia are then prepared as in step a of Scheme II by coupling a compound of Formula VIb, where B(O)₂R14 is as previously defined, with a compound of Formula VII, where R6 is as previously defined and Y is a halogen using a base such as cesium carbonate or sodium carbonate and a catalyst, such as 1,1′-bis(diphenylphosphino)ferrocenepalladium(II) dichloride or tetrakis(triphenylphosphine)palladium(0) in a solvent such as toluene or 1,4-dioxane.

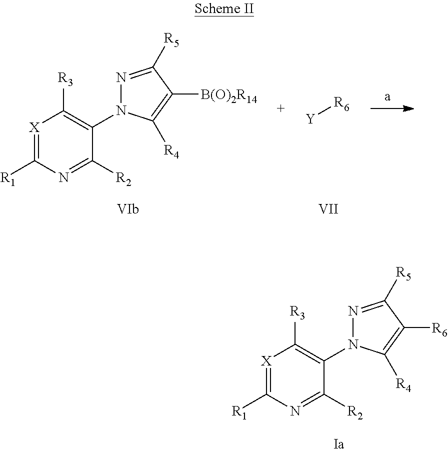

Compounds of Formula Ib can be prepared according to step a in Scheme III by coupling a compound of Formula Vb, where Y is a halogen, with a compound of Formula VIII, where R6 and B(O)₂R14 are as previously defined, and using a base such as cesium carbonate and a catalyst, such as 1,1′-bis(triphenylphosphine)palladium(II) chloride or 1,1′-Bis(diphenylphosphino)ferrocene]dichloropalladium(II) in a solvent system such as acetonitrile or 1,4-Dioxane with water.

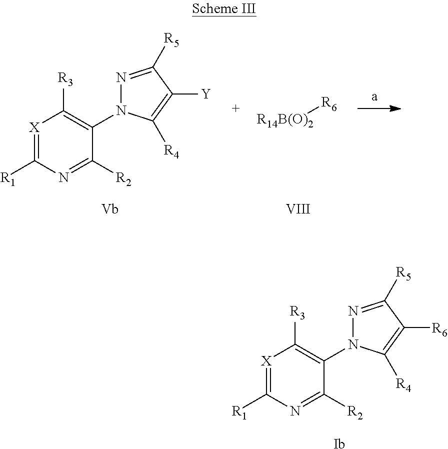

Preparation of compounds of Formula VIc, where B(O)₂R14 is a boronic acid or boronic ester and R4 is Cl is demonstrated in Scheme IV. The compound of Formula X in step a, where in R4 is Cl, R5 is H and X⁻ represents Cl⁻, can be prepared according to the methods described in Acta. Pharm. Suec. 22, 147-156 (1985) by Tolf, Bo-Ragnar and Dahlbom, R. Alternatively, in step a of Scheme IV compounds of the Formula X, wherein R4 is Cl, X⁻ represents Cl⁻ and R5 is as defined previously, can be prepared by treating compounds of the Formula IX, wherein R5 is as defined previously, with a hydrosilane such as triethyl silane in the presence of a metal catalyst such as 5% Pd on alumina and an acid such as HCl or HBr, respectively, in a solvent such as ethanol. The compound of Formula XII in step b can be obtained via aryl nucleophilic substitution of Formula XI where W is fluorine, in the presence of a pyrazole of Formula X with a base such as Sodium hydride in a polar aprotic solvent such as DMF or DMSO. In step c, following the procedure of Mo, Fanyang et al. Angewandte Chemie International Edition, 49: 1846-1849 compounds of Formula VIc, where B(O)₂R14 is a boronic ester, can be prepared by reacting compounds of formula XII with t-butyl nitrite in the presents of bis(pinacolato)diboron and benzoyl peroxide in a solvent such as acetonitrile.

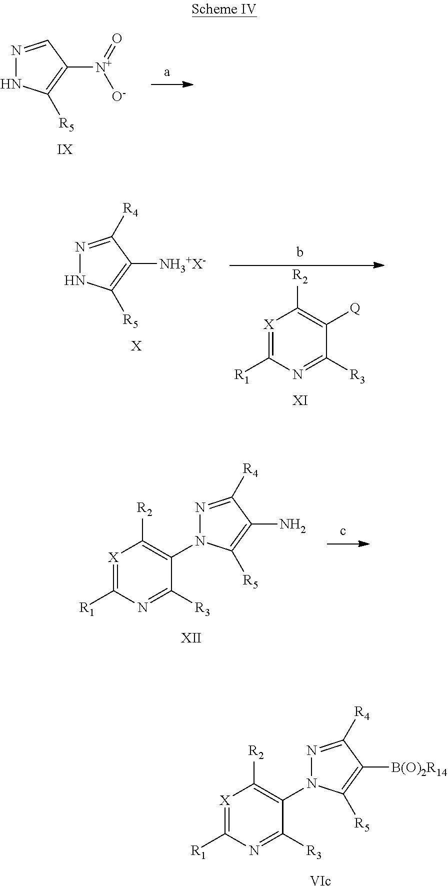

### Examples

The examples are for illustration purposes and are not to be construed as limiting the invention disclosed in this document to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Molecules are given their known names, named according to naming programs within ISIS Draw, ChemDraw or ACD Name Pro. If such programs are unable to name a molecule, the molecule is named using conventional naming rules. ¹H NMR spectral data are in ppm (δ) and were recorded at 300, 400 or 600 MHz, and ¹³C NMR spectral data are in ppm (δ) and were recorded at 75, 100 or 150 MHz, unless otherwise stated.

### Example 1

### Preparation of 3-(4-(4,4,5,5-tetramethyl-1,3,2-dioxaborolan-2-yl)-1H-pyrazol-1-yl)pyridine

### Example 1

### Step 1: Preparation of 3-(1H-pyrazol-1-yl)pyridine

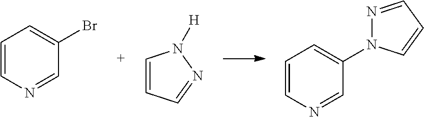

To a solution of copper(I) oxide (0.906 g, 6.33 mmol), salicylaldoximine (3.47 g, 25.3 mmol), 1H-pyrazole (12.93 g, 190 mmol), and cesium carbonate (66.0 g, 203 mmol) in acetonitrile (50.6 ml) under a nitrogen stream was added 3-bromopyridine (20 g, 127 mmol). The reaction mixture was heated at reflux for 24H. The reaction mixture was allowed to cool to ambient temperature, and then diluted with EtOAc, filtered through celite, and washed with H₂O and saturated brine solution. The organics were separated, dried with MgSO₄, filtered and concentrated in vacuo. The residue was purified by column chromatography eluting with 0-50% acetone in hexanes to afford 3-(1H-pyrazol-1-yl)pyridine as a yellow oil (17 g, 93%): ¹H NMR (400 MHz, acetone-d₆) δ 9.14 (d, J=2.2 Hz, 1H), 8.54 (d, J=3.8 Hz, 1H), 8.45 (dd, J=2.5, 0.5 Hz, 1H), 8.24 (ddd, J=8.3, 2.7, 1.5 Hz, 1H), 7.79 (d, J=1.5 Hz, 1H), 7.53 (ddd, J=8.3, 4.7, 0.7 Hz, 1H), 6.59 (dd, J=2.5, 1.8 Hz, 1H); EIMS m/z 145.

### Example 1

### Step 2: Preparation of 3-(4-bromo-1H-pyrazol-1-yl)pyridine

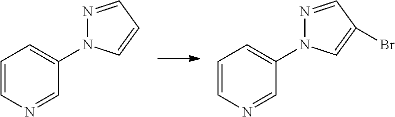

To a solution of 3-(1H-pyrazol-1-yl)pyridine (5 g, 34.4 mmol) in acetonitrile (68.9 ml) was added N-bromosuccinimide (7.97 g, 44.8 mmol). The reaction mixture was stirred at ambient temperature for 3H. The reaction mixture was diluted with EtOAc and washed with H₂O, the organics were dried with MgSO₄, filtered and concentrated in vacuo. The residue was purified by column chromatography eluting with 0-60% EtOAc in hexanes to afford 3-(4-bromo-1H-pyrazol-1-yl)pyridine as a tan solid (6.99 g, 91%): mp 126-127° C.; ¹H NMR (400 MHz, acetone-d₆) δ 9.12 (d, J=2.5 Hz, 1H), 8.64 (d, J=0.5 Hz, 1H), 8.58 (dd, J=4.7, 1.4 Hz, 1H), 8.23 (ddd, J=8.3, 2.7, 1.4 Hz, 1H), 7.85 (s, 1H), 7.56 (ddd, J=8.3, 4.7, 0.7 Hz, 1H); EIMS m/z 223.

### Example 1

### Step 3: Preparation of 3-(4-(4,4,5,5-tetramethyl-1,3,2-dioxaborolan-2-yl)-1H-pyrazol-1-yl)pyridine

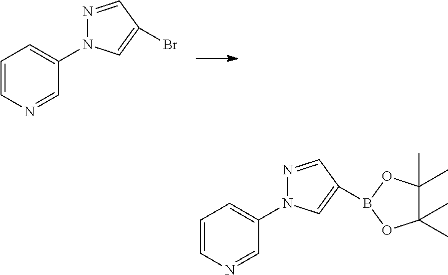

To a solution of 3-(4-bromo-1H-pyrazol-1-yl)pyridine (1 g, 4.46 mmol) in N,N-dimethylformamide (11.16 ml) was added potassium acetate (2.190 g, 22.32 mmol), bis(pinacolato)diboron (3.40 g, 13.39 mmol), and 1,1′-bis(diphenylphosphino)ferrocenepalladium(II) dichloride (0.367 g, 0.446 mmol). The reaction mixture was heated at 80° C. for 18H. The reaction mixture was diluted with Et₂O and was washed with H₂O and brine solution. The organics were dried with MgSO₄, filtered and concentrated in vacuo. The residue was purified by column chromatography eluting with 0-70% acetone in dichloromethane to afford 3-(4-(4,4,5,5-tetramethyl-1,3,2-dioxaborolan-2-yl)-1H-pyrazol-1-yl)pyridine as a brown solid (292 mg, 24%): ¹H NMR (400 MHz, acetone-d₆) δ 9.20 (d, J=2.3 Hz, 1H), 8.59 (d, J=0.5 Hz, 1H), 8.57 (dd, J=4.7, 1.4 Hz, 1H), 8.30 (ddd, J=8.3, 2.7, 1.5 Hz, 1H), 7.91 (s, 1H), 7.55 (ddd, J=8.3, 4.7, 0.7 Hz, 1H), 1.34 (s, 12H); EIMS m/z 271.

### Example 2

### Preparation of 3-(4-iodo-1H-pyrazol-1-yl)pyridine

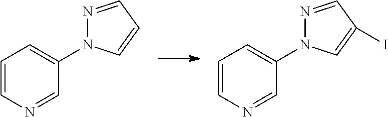

To a mixture of 3-(1H-pyrazol-1-yl)pyridine (5 g, 34.4 mmol), iodic acid (1.212 g, 6.89 mmol), and iodine (3.50 g, 13.78 mmol) in acetic acid (49.2 ml) was added sulfuric acid (0.918 ml, 17.22 mmol). The reaction mixture heated to 70° C. for 30 min. The reaction mixture was poured onto ice water, extracted with Et₂O and then was washed with sodium thiosulfate solution. The organics were then dried with MgSO4, filtered and concentrated in vacuo. The residue purified by flash chromatography eluting with 0-50% acetone in dichloromethane to yielded a light tan solid (6.1 g, 21.38 mmol, 62.1% yield): mp 145-146° C.; ¹H NMR (400 MHz, acetone-d₆) δ 9.11 (d, J=2.3 Hz, 1H), 8.60 (d, J=0.5 Hz, 1H), 8.56 (dd, J=4.7, 1.4 Hz, 1H), 8.22 (ddd, J=8.3, 2.7, 1.5 Hz, 1H), 7.84 (s, 1H), 7.54 (ddd, J=8.3, 4.7, 0.7 Hz, 1H); EIMS m/z 271.

### Example 3

### Preparation of 2-(1-(pyridin-3-yl)-1H-pyrazol-4-yl)-6-(trifluoromethyl)pyridine (Compound 1)

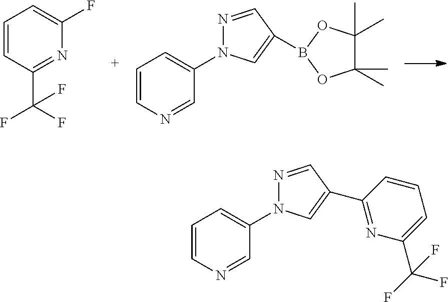

To a solution of 2-bromo-6-(trifluoromethyl)pyridine (200 mg, 0.885 mmol) in a solvent mixture of toluene (1.5 ml)/water (1.5 ml)/ethanol (0.7 ml) was added 3-(4-(4,4,5,5-tetramethyl-1,3,2-dioxaborolan-2-yl)-1H-pyrazol-1-yl)pyridine (200 mg, 0.738 mmol), cesium carbonate (601 mg, 1.844 mmol) and 1,1′-bis(diphenylphosphino)ferrocenepalladium(II) dichloride (30.3 mg, 0.037 mmol). The reaction mixture was heated in a benchtop microwave at 120° C. for 30 min. The reaction mixture was diluted with water and extracted with EtOAc. The organics were dried with MgSO₄, filtered and then concentrated in vacuo. The residue was purified by column chromatography, eluting with 0-50% acetone in hexanes to afford 2-(1-(pyridin-3-yl)-1H-pyrazol-4-yl)-6-(trifluoromethyl)pyridine as a white solid (82 mg, 38.3%): mp 130-131° C.; ¹H NMR (400 MHz, acetone-d₆) δ 9.24 (d, J=2.4 Hz, 1H), 9.13 (d, J=0.5 Hz, 1H), 8.60 (dd, J=4.7, 1.4 Hz, 1H), 8.44 (s, 1H), 8.35 (ddd, J=8.3, 2.7, 1.5 Hz, 1H), 8.12 (qd, J=8.3, 4.7 Hz, 2H), 7.72 (dd, J=7.1, 1.5 Hz, 1H), 7.59 (ddd, J=8.3, 4.7, 0.7 Hz, 1H); EIMS m/z 290.

### Example 4

### Preparation of 5-(1-(pyridin-3-yl)-1H-pyrazol-4-yl)-2-(trifluoromethyl)pyridine (Compound 2)

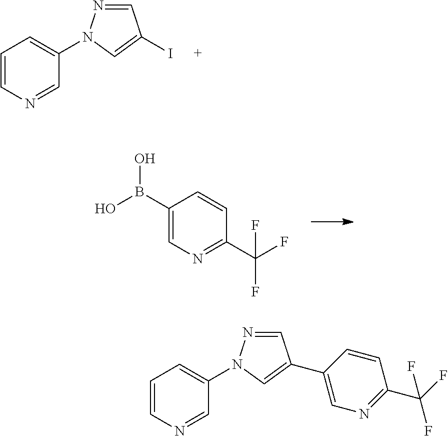

To a mixture of 3-(4-iodo-1H-pyrazol-1-yl)pyridine (200 mg, 0.738 mmol) in acetonitrile (1 ml) and water (1 ml) was added 6-(trifluoromethyl)pyridin-3-ylboronic acid (211 mg, 1.107 mmol), cesium carbonate (481 mg, 1.476 mmol), and bis(triphenylphosphine)palladium(II) chloride (51.8 mg, 0.074 mmol). The reaction mixture was then heated at 120° C. for 30 min in a Biotage bench top microwave. The reaction mixture was then diluted with dichloromethane and washed with H₂O. The phases were separated and the organics were concentrated. The residue was purified by flash chromatography eluted with 0-50% acetone in hexanes to yield 5-(1-(pyridin-3-yl)-1H-pyrazol-4-yl)-2-(trifluoromethyl)pyridine as a white solid (110 mg, 0.360 mmol, 48.8% yield): mp 203-204° C.; ¹H NMR (400 MHz, acetone-d₆) δ 9.21 (d, J=2.3 Hz, 1H), 9.18 (dd, J=2.9, 1.4 Hz, 2H), 8.61 (dd, J=4.7, 1.4 Hz, 1H), 8.44 (d, J=0.4 Hz, 1H), 8.42-8.37 (m, 1H), 8.32 (ddd, J=8.3, 2.7, 1.5 Hz, 1H), 7.91 (dd, J=8.2, 0.6 Hz, 1H), 7.60 (ddd, J=8.3, 4.7, 0.7 Hz, 1H); EIMS m/z 290.

### Example 5

### Preparation of 3-fluoro-5-(3-methyl-1H-pyrazol-1-yl)pyridine

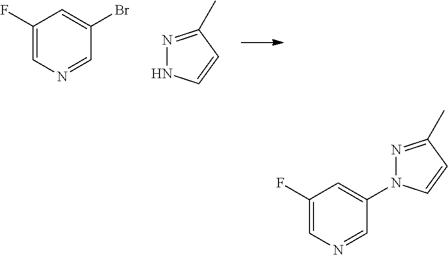

An oven dried round bottom flask evacuated and back-fill with dry nitrogen was charged with copper(I) oxide (0.020 g, 0.142 mmol), (E)-2-hydroxybenzaldehyde oxime (0.078 g, 0.568 mmol), 3-methyl-1H-pyrazole (0.350 g, 4.26 mmol), cesium carbonate (1.48 g, 4.55 mmol), and 3-bromo-5-fluoropyridine (5 g, 28.4 mmol) was added under a stream of nitrogen followed by acetonitrile (11.36 mL). The reaction mixture was stirred overnight at 82° C. The reaction was then filtered through celite, and the celite was washed with DCM. The mother liquor was washed with water and brine, concentrated and purified by normal phase chromatography eluting with 0-75% ethyl acetate in hexanes to give the title compound as an off-white solid (170 mg, 34%): mp 70-72° C.; ¹H NMR (400 MHz, CDCl₃) δ 8.75 (m, 1H), 8.35 (d, J=2.6 Hz, 1H), 7.87 (m, 1H), 7.81 (m, 1H), 6.31 (d, J=2.5 Hz, 1H), 2.37 (s, 3H); EIMS m/z 177.

### Example 6

### Preparation of 3-(3-methyl-1H-pyrazol-1-yl)pyridine

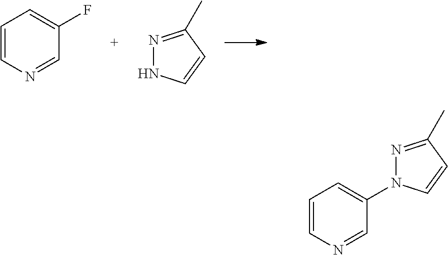

To a solution of 60% sodium hydride in mineral oil (0.494 g, 12.36 mmol) in dry DMSO (20.60 mL) was added 3-methyl-1H-pyrazole (1.015 g, 12.36 mmol) slowly under a N₂. This was stirred at ambient temp until no bubbling was observed (1 hr). To this mixture was added 3-fluoropyridine (1.0 g, 10.30 mmol), and then the reaction mixture was heated at 100° C. for 3 hr. The reaction mixture was allowed to cool to ambient temperature and then, it was diluted with H₂O and extracted with DCM. The organic phase was dried with MgSO₄, filtered and then the solvent was removed in vacuo. The residual oil was purified by normal phase chromatography eluting with 0-60% acetone in hexanes to afford 3-(3-methyl-1H-pyrazol-1-yl)pyridine as a white solid (828 mg, 50%): ¹H NMR (400 MHz, CDCl₃) δ 8.97-8.91 (m, 1H), 8.50 (dd, J=4.7, 1.4 Hz, 1H), 8.01 (ddd, J=8.3, 2.7, 1.5 Hz, 1H), 7.85 (d, J=2.4 Hz, 1H), 7.37 (ddd, J=8.3, 4.7, 0.7 Hz, 1H), 6.30 (d, J=2.4 Hz, 1H), 2.39 (s, 3H); ESIMS m/z 159 ([M+H]⁺).

### Example 7

### Preparation of 3-fluoro-5-(4-iodo-3-methyl-1H-pyrazol-1-yl)pyridine

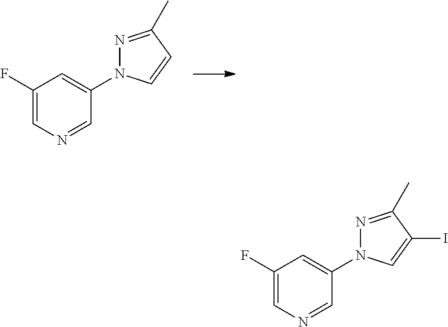

To a mixture of 3-fluoro-5-(3-methyl-1H-pyrazol-1-yl)pyridine (1.9 g, 10.72 mmol), iodic acid (0.755 g, 4.29 mmol), and iodine (2.177 g, 8.58 mmol) in acetic acid (15.32 mL) was added conc. sulfur acid (0.953 ml, 5.36 mmol). The reaction mixture heated to 70° C. for 30 min. The reaction mixture was cooled to ambient temperature, and then poured onto ice with sodium thiosulfate. This mixture was extracted with Et₂O (3×), and then the organics were combined. The organics were wash with sat. NaHCO₃ solution, dried with MgSO4, filtered and concentrated in vacuo. The crude product was purified by normal phase chromatography eluting with 0-50% acetone in hexanes to yield the title compound as a white solid (2.74 g, 83%): mp 87-88° C.; ¹H NMR (400 MHz, acetone-d₆) δ 8.97 (dd, J=1.7, 1.1 Hz, 1H), 8.58-8.53 (m, 1H), 8.47-8.42 (m, 1H), 8.08-7.99 (m, 1H), 2.30 (s, 3H); EIMS m/z 303.

### Example 8

### Preparation of 3-fluoro-5-(3-methyl-4-(4,4,5,5-tetramethyl-1,3,2-dioxaborolan-2-yl)-1H-pyrazol-1-yl)pyridine

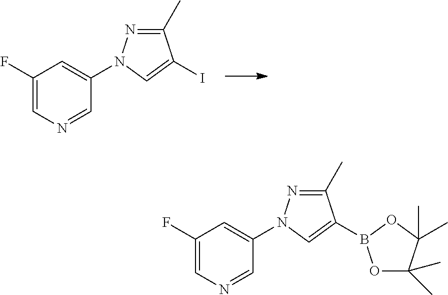

To a solution of 3-fluoro-5-(4-iodo-3-methyl-1H-pyrazol-1-yl)pyridine (1.75 g, 5.77 mmol) in dry THF (11.55 mL) cooled to 0° C. under a N₂ atmosphere was added 2M isopropylmagnesium chloride in THF (3.32 mL, 6.64 mmol). The reaction mixture was stirred for 1 hr at 0° C. Then 2-Isopropoxy-4,4,5,5-tetramethyl-1,3,2-dioxaborolane (1.611 g, 8.66 mmol) was added and the reaction mixture was allowed to warm to ambient temperature overnight. The reaction was then quench with sat. NH₄Cl solution. The resulting mixture was extracted with EtOAc(3×), organic were combined dried with MgSO4, filtered and concentrated. in vacuo. The residue was dissolved in Et₂O and extracted 3× with 1M NaOH. The aqueous layer was then was acidified with 2M HCl and extracted (3×) with Et₂O. The organics were combined, dried with MgSO4, filtered and concentrated in vacuo to yielded as a white solid (1.16 g, 66.3%): mp 109-110° C., ¹H NMR (400 MHz, acetone-d₆) δ 9.06 (m, 1H), 8.53 (s, 1H), 8.45 (m, 1H), 8.13 (m, 1H), 2.42 (s, 3H); EIMS m/z 303.

### Example 9

### Preparation of 3-(3-methyl-4-(4,4,5,5-tetramethyl-1,3,2-dioxaborolan-2-yl)-1H-pyrazol-1-yl)pyridine

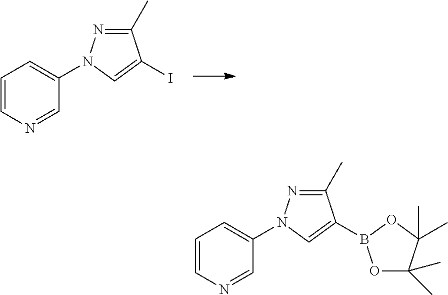

To a solution of 3-(4-iodo-3-methyl-1H-pyrazol-1-yl)pyridine (5 g, 17.54 mmol) in THF (35.1 mL) cooled to 0° C. under a N₂ atmosphere was added 2 M isopropylmagnesium chloride in THF (10.08 mL, 20.17 mmol). The reaction mixture was stirred for 1 hr at 0° C. Then 2-isopropoxy-4,4,5,5-tetramethyl-1,3,2-dioxaborolane (4.89 g, 26.3 mmol) was added, and then reaction mixture was allowed to warm to ambient temp overnight. The reaction was quench with sat. NH₄Cl solution then extracted with EtOAc (3×), The organics were combined dried with MgSO4, filtered and concentrated. The residue was dissolved in Et₂O and extracted 3× with 1M NaOH. The aqueous layer was then was acidified with 2M HCl and extracted 3× with Et₂O. The organics were combined, dried with MgSO4, filtered and concentrated in vacuo to afford a brown oil (4.5 g, 82%): ¹H NMR (400 MHz, CDCl₃) δ 8.96 (dd, J=2.6, 0.5 Hz, 1H), 8.51 (dd, J=4.7, 1.5 Hz, 1H), 8.20-8.15 (m, 1H), 8.02 (ddd, J=8.3, 2.7, 1.5 Hz, 1H), 7.37 (ddd, J=8.3, 4.8, 0.7 Hz, 1H), 1.34 (s, 13H); EIMS m/z 285.

### Example 10

### Preparation of 2-(1-(5-fluoropyridin-3-yl)-3-methyl-1H-pyrazol-4-yl)-6-(trifluoromethyl)pyridine (Compound 3)

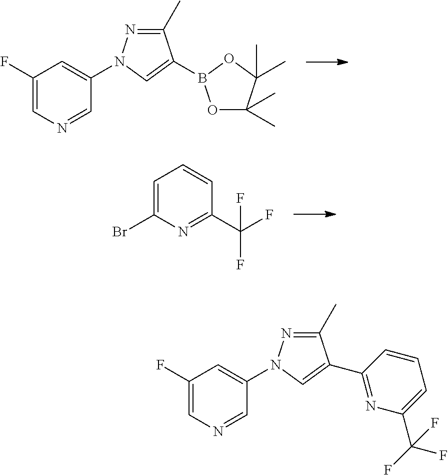

To a solution of 3-fluoro-5-(3-methyl-4-(4,4,5,5-tetramethyl-1,3,2-dioxaborolan-2-yl)-1H-pyrazol-1-yl)pyridine (322 mg, 1.062 mmol) in 1,4-dioxane (2.95 mL) was added 2-bromo-6-(trifluoromethyl)pyridine (200 mg, 0.885 mmol), 2M Na₂CO₃ (1.770 ml, 3.54 mmol) and tetrakis(triphenylphosphine)palladium(0) (51.1 mg, 0.044 mmol). The reaction mixture was heated in a bench top microwave at 120° C. for 40 min. The reaction mixture was then diluted with DCM and washed with H₂O. The phases were separated with a Biotage Phase separator and the organic phase was concentrated. The residue was purified by normal phase chromatography eluting with 0-40% acetone in hexanes to afford a white solid (263 mg, 88%): mp 130-131° C., ¹H NMR (400 MHz, acetone-d₆) δ 9.15-9.09 (m, 1H), 9.09-9.04 (m, 1H), 8.50-8.44 (m, 1H), 8.16-8.09 (m, 2H), 8.03 (d, J=7.8 Hz, 1H), 7.71 (dd, J=7.6, 0.8 Hz, 1H), 2.73-2.62 (m, 3H); EIMS m/z 322.

Compounds 4-5, and 7-13 were prepared in accordance with the procedures disclosed in Example 10.

### Example 11

### Preparation of 3-(3-methyl-4-(3-(methylthio)phenyl)-1H-pyrazol-1-yl)pyridine (Compound 17)

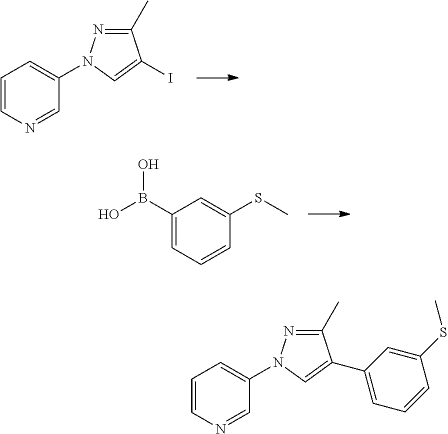

To a mixture of 3-(4-iodo-3-methyl-1H-pyrazol-1-yl)pyridine (100 mg, 0.7042 mmol) and (3-(methylthio)phenyl)boronic acid (141.9 mg, 0.845 mmol) in 1-4 dioxane:water (2:1) (4.5 mL) was added cesium carbonate (504.7 mg, 1.5492 mmol), purged with Argon gas for 5-10 min then PdCl2(dppf) (51.5 mg, 0.0704 mmol) was added again purged with Argon gas for 5-10 min. The reaction mixture was then heated at 100° C. for 16 hrs and cooled to room temperature then filtered, diluted with ethyl acetate and washed with H₂O. The organic layer was concentrated under reduced pressure. The residue was purified by Prep HPLC to afford a grey solid (78 mg, 40%): mp 104-109° C.; ¹H NMR (300 MHz, CDCl₃) δ 8.99 (d, J=2.5 Hz, 1H), 8.53 (dd, J=4.7, 1.3 Hz, 1H), 8.06 (ddd, J=8.3, 2.6, 1.5 Hz, 1H), 8.00 (s, 1H), 7.37 (m, 3H), 7.23 (m, 2H), 2.53 (s, 3H), 2.49 (s, 3H); ESIMS m/z 281 ([M+H]⁺).

Compounds 23, and 25-30 were prepared in accordance with the procedures disclosed in Example 11. Compounds 19, 21 and 22 were made in accordance with the procedure disclosed in Example 11, from 3-methyl-1-(pyridin-3-yl)-1H-pyrazol-4-ylboronic acid and the appropriately substituted halo-phenyl reagent.

### Example 12

### Preparation of 3-chloro-1H-pyrazol-4-amine hydrochloride

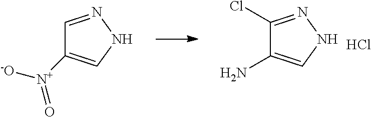

Into a 2 L three-necked round bottom flask affixed with an overhead stirrer, a temperature probe, an addition funnel, and a nitrogen inlet were added ethanol (600 mL) and 4-nitro-1H-pyrazole (50.6 g, 447 mmol). To this solution was added, in one portion, conc. HCl (368 mL) (note: rapid exotherm from 15° C. to 39° C.) and the resulting mixture was purged with nitrogen for 5 minutes. Palladium on alumina (5% w/w) (2.6 g, Alfa, black solid) was added to the mixture and stirred at room temperature while triethylsilane (208 g, 1789 mmol) was added drop-wise over 4 h. The reaction, which started to slowly exotherm from 35° C. to 55° C. over 2.0 h, was stirred for a total of 16 h and vacuum filtered through a plug of Celite® to give a biphasic mixture. The mixture was transferred to a separatory funnel, the bottom aqueous layer was collected and rotary evaporated (60° C., 50 mmHg) to dryness with the aid of acetonitrile (3×350 mL). The resulting yellow solid was suspended in acetonitrile (150 mL) and allowed to stand for 2 h at room temperature followed by 1 h at 0° C. in the refrigerator. The solids were filtered and washed with acetonitrile (100 mL) to afford the titled compound 3-chloro-1H-pyrazol-4-amine hydrochloride (84 g, 97% yield, 80% purity) as a white solid: mp 190-193° C.; ¹H NMR (400 MHz, DMSO-d₆) δ 10.46-10.24 (bs, 2H), 8.03 (s, 0.54H), 7.75 (s, 0.46H), 5.95 (bs, 1H); ¹³C NMR (101 MHz, DMSO) δ 128.24, 125.97, 116.71.

### Example 13

### Preparation of 3-chloro-1-(5-fluoropyridin-3-yl)-1H-pyrazol-4-amine

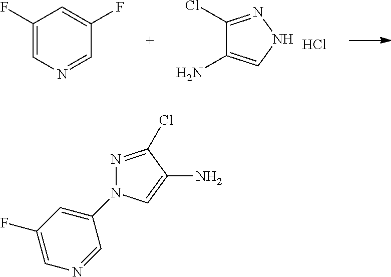

To a stirred solution of 5-chloro-1H-pyrazol-4-amine, HCl (2 g, 12.99 mmol) and cesium carbonate (8.89 g, 27.3 mmol) in DMF (13 mL) was added 3,5-difluoropyridine (1.794 g, 15.58 mmol) and the mixture heated at 70° C. for 12 h. The mixture was cooled to room temperature and filtered. The solids were washed with copious amount of ethyl acetate. The filtrates was washed with brine, dried over anhydrous MgSO₄ and concentrated in vacuo to give a brown solid. This solid was dissolved in ethyl acetate and the resulting solution was saturated with hexanes to precipitate 3-chloro-1-(5-fluoropyridin-3-yl)-1H-pyrazol-4-amine (2.31 g, 10.32 mmol, 79%) as a brown solid: ¹H NMR (400 MHz, DMSO-d₆) δ 8.89-8.82 (m, 1H), 8.45 (d, J=2.5 Hz, 1H), 8.07 (d, J=10.4 Hz, 1H), 7.94 (s, 1H), 4.51 (s, 2H); EIMS (m/z) 213 ([M+1]+).

### Example 14

### Preparation of 3-(3-chloro-4-(4,4,5,5-tetramethyl-1,3,2-dioxaborolan-2-yl)-1H-pyrazol-1-yl)-5-fluoropyridine

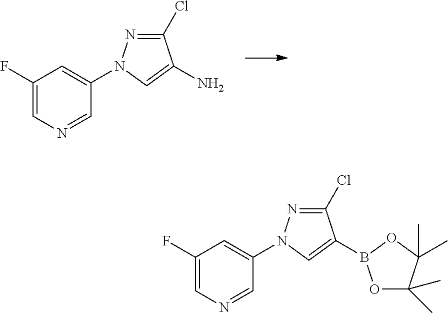

To a solution of 3-chloro-1-(5-fluoropyridin-3-yl)-1H-pyrazol-4-amine (800 mg, 3.76 mmol) in acetonitrile (9.407 mL) in an ice bath was added bis(pinacolato)diboron (1433 mg, 5.64 mmol) and benzoic peroxyanhydride (182 mg, 0.753 mmol) followed by dropwise addition of t-Butyl nitrite (0.537 ml, 4.52 mmol) (exothermic). The reaction mixture was allowed to warm to ambient temperature and stirred overnight. The reaction mixture was concentrated and purified by normal phase chromatography eluting with 0-100% acetone in hexanes to afford a tan solid (121 mg, 10%): mp 125° C.; ¹H NMR (400 MHz, acetone-d₆) δ 9.08-9.02 (m, 1H), 8.67 (s, 1H), 8.52 (dd, J=2.5, 0.4 Hz, 1H), 8.20-8.11 (m, 1H), 1.34 (s, 13H); ELMS m/z 323.

### Example A

### Bioassays on Beet Armyworm (“BAW”) and Corn Earworm (“CEW”)

BAW has few effective parasites, diseases, or predators to lower its population. BAW infests many weeds, trees, grasses, legumes, and field crops. In various places, it is of economic concern upon asparagus, cotton, corn, soybeans, tobacco, alfalfa, sugar beets, peppers, tomatoes, potatoes, onions, peas, sunflowers, and citrus, among other plants. CEW is known to attack corn and tomatoes, but it also attacks artichoke, asparagus, cabbage, cantaloupe, collards, cowpeas, cucumbers, eggplant, lettuce, lima beans, melon, okra, peas, peppers, potatoes, pumpkin, snap beans, spinach, squash, sweet potatoes, and watermelon, among other plants. CEW is also known to be resistant to certain insecticides. Consequently, because of the above factors, control of these pests is important. Furthermore, molecules that control these pests are useful in controlling other pests.

Certain molecules disclosed in this document were tested against BAW and CEW using procedures described in the following examples. In the reporting of the results, the “Table 3: BAW & CEW Rating Table” was used (See Table Section).

Bioassays on BAW (Spodoptera exigua)

Bioassays on BAW were conducted using a 128-well diet tray assay. One second instar BAW larva was placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays were covered with a clear self-adhesive cover, and held at 25° C., 14:10 light-dark for five days. Percent mortality was recorded for the larvae in each well; activity in the eight wells was then averaged. The results are indicated in the table entitled “Table 5: BAW, CEW, GPA, Scale and WF Results” (See Table Section).

Bioassays on CEW (Helicoverpa zea)

Bioassays on CEW were conducted using a 128-well diet tray assay. One second instar CEW larvae was placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays were covered with a clear self-adhesive cover, and held at 25° C., 14:10 light-dark for five days. Percent mortality was recorded for the larvae in each well; activity in the eight wells was then averaged. The results are indicated in the table entitled “Table 5: BAW, CEW, GPA, Scale and WF Results” (See Table Section).

### Example B

### Bioassays on Green Peach Aphid (“GPA”) (Myzus persicae)

GPA is the most significant aphid pest of peach trees, causing decreased growth, shriveling of the leaves, and the death of various tissues. It is also hazardous because it acts as a vector for the transport of plant viruses, such as potato virus Y and potato leafroll virus to members of the nightshade/potato family Solanaceae, and various mosaic viruses to many other food crops. GPA attacks such plants as broccoli, burdock, cabbage, carrot, cauliflower, daikon, eggplant, green beans, lettuce, macadamia, papaya, peppers, sweet potatoes, tomatoes, watercress, and zucchini, among other plants. GPA also attacks many ornamental crops such as carnation, chrysanthemum, flowering white cabbage, poinsettia, and roses. GPA has developed resistance to many pesticides.

Certain molecules disclosed in this document were tested against GPA using procedures described in the following example. In the reporting of the results, the “Table 4: GPA, Scale and WF Rating Table” was used (See Table Section).

Cabbage seedlings grown in 3-inch pots, with 2-3 small (3-5 cm) true leaves, were used as test substrate. The seedlings were infested with 20-50 GPA (wingless adult and nymph stages) one day prior to chemical application. Four pots with individual seedlings were used for each treatment. Test compounds (2 mg) were dissolved in 2 mL of acetone/methanol (1:1) solvent, forming stock solutions of 1000 ppm test compound. The stock solutions were diluted 5× with 0.025% Tween 20 in H₂O to obtain the solution at 200 ppm test compound. A hand-held aspirator-type sprayer was used for spraying a solution to both sides of cabbage leaves until runoff. Reference plants (solvent check) were sprayed with the diluent only containing 20% by volume of acetone/methanol (1:1) solvent. Treated plants were held in a holding room for three days at approximately 25° C. and ambient relative humidity (RH) prior to grading. Evaluation was conducted by counting the number of live aphids per plant under a microscope. Percent Control was measured by using Abbott's correction formula (W. S. Abbott, “A Method of Computing the Effectiveness of an Insecticide” J. Econ. Entomol. 18 (1925), pp. 265-267) as follows.

Corrected % Control=100*(X−Y)/X
where
X=No. of live aphids on solvent check plants and
Y=No. of live aphids on treated plants

The results are indicated in the table entitled “Table 5: BAW, CEW, GPA, Scale and WF Results” (See Table Section).

### Example C

### Bioassays on Sweet Potato Whitefly (“WF”) (Bemisia tabaci)

Sweet potato whitefly (Bemisia tabaci) has been reported as a serious pest of cultivated crops world-wide. It has an extremely wide host range attacking more than 500 species of plants from 63 plant families Weeds often serve as alternate hosts of crop pests. Direct feeding damage is caused by the piercing and sucking sap from the foliage of plants. This feeding causes weakening and early wilting of the plant and reduces the plant growth rate and yield. Indirect damage results by the accumulation of honeydew produced by the whiteflies. Honeydew serves as a substrate for the growth of black sooty mold on leaves and fruit reducing photosynthesis and lessens the market value of the plant or yield. Damage is also caused when sweet potato whitefly vectors plant viruses. The sweet potato whitefly is considered the most common and important whitefly vector of plant viruses worldwide.

Certain molecules disclosed in this document were tested against WF using procedures described in the following example. In the reporting of the results, the “Table 4: GPA, Scale and WF Rating Table” was used (See Table Section).

Cotton plants (Gossypium hirsutum) grown in 3-inch pots, with 1 small (4-5 cm) true leaves, were used as test substrate. The plants were infested with 200-400 whitefly eggs 4-5 days prior to chemical application. Four pots with individual plants were used for each treatment. Test compounds (2 mg) were dissolved in 1 mL of acetone solvent, forming stock solutions of 2000 ppm test compound. The stock solutions were diluted 10× with 0.025% Tween 20 in H₂O (diluents) to obtain the solution at 200 ppm test compound. A hand-held aspirator-type sprayer was used for spraying a solution to both sides of cotton leaves until runoff. Reference plants (solvent check) were sprayed with the diluent only containing 10% by volume of acetone solvent. Treated plants were held in a holding room for 9 days at approximately 25° C. and ambient relative humidity (RH) prior to grading. Evaluation was conducted by counting the number of live 3-4 nymphal stage per plant under a microscope. Percent Control was measured by using Abbott's correction formula (W. S. Abbott, “A Method of Computing the Effectiveness of an Insecticide” J. Econ. Entomol. 18 (1925), pp. 265-267) as follows.

Corrected % Control=100*(X−Y)/X
where
X=No. of live nymphs on solvent check plants and
Y=No. of live nymphs on treated plants.

The results are indicated in the table entitled “Table 5: BAW, CEW, GPA, Scale and WF Results” (See Table Section).

### Example D

### Bioassays on San Jose Scale (“Scale”) (Quadraspidiotus perniciosus)

San Jose scale (Quadraspidiotus perniciosus) is a key pest in almost all the fruit growing districts of the United States. It was introduced to California from China on flowering peach in the early 1870s and soon became a serious pest in the San Jose area. By the late 1890s it had spread to all parts of the United States. This was the first insect to develop resistance to a pesticide in the U.S. (to lime sulfur in 1908) and has been responsible for the death of thousands of acres of apples since it was introduced. A single female and her offspring can produce several thousand scales in one season and they can kill the tree as well as make the fruit unmarketable. The pest has become of increasing concern to the Northwest tree fruit industry due to the importance of exports, as phytosanitary regulations bar infested fruit from some countries.

Certain molecules disclosed in this document were tested against Scale using procedures described in the following example. In the reporting of the results, the “Table 4: GPA, Scale and WF Rating Table” was used (See Table Section).

Cotton plants (Gossypium hirsutum) grown in 3-inch pots, with 1 small (4-5 cm) true leaves, were used as test substrate. The plants were infested with 200-400 scale crawlers 1-2 hours after chemical application. Four pots with individual plants were used for each treatment. Test compounds (2 mg) were dissolved in 1 mL of acetone solvent, forming stock solutions of 2000 ppm test compound. The stock solutions were diluted 10× with 0.025% Tween 20 in H₂O (diluents) to obtain the solution at 200 ppm test compound. A hand-held aspirator-type sprayer was used for spraying a solution to both sides of cotton leaves until runoff. Reference plants (solvent check) were sprayed with the diluent only containing 10% by volume of acetone solvent. Treated plants were held in a holding room for three days at approximately 25° C. and ambient relative humidity (RH) prior to grading. Evaluation was conducted by counting the number of live 2-3 nymphal stage per plant under a microscope 9 days after infestation. Percent Control was measured by using Abbott's correction formula (W. S. Abbott, “A Method of Computing the Effectiveness of an Insecticide” J. Econ. Entomol. 18 (1925), pp. 265-267) as follows.

Corrected % Control=100*(X−Y)/X
where
X=No. of live nymphs on solvent check plants and
Y=No. of live nymphs on treated plants

The results are indicated in the table entitled “Table 5: BAW, CEW, GPA, Scale and WF Results” (See Table Section).

### Pesticidally Acceptable Acid Addition Salts, Salt Derivatives, Solvates, Polymorphs, Isotopes and Radionuclides

Molecules of Formula One may be formulated into pesticidally acceptable acid addition salts. By way of a non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxymethanesulfonic, and hydroxyethanesulfonic acids. Additionally, by way of a non-limiting example, an acid function can form salts including those derived from alkali or alkaline earth metals and those derived from ammonia and amines. Examples of preferred cations include sodium, potassium, and magnesium.

Molecules of Formula One may be formulated into salt derivatives. By way of a non-limiting example, a salt derivative can be prepared by contacting a free base with a sufficient amount of the desired acid to produce a salt. A free base may be regenerated by treating the salt with a suitable dilute aqueous base solution such as dilute aqueous sodium hydroxide (NaOH), potassium carbonate, ammonia, and sodium bicarbonate. As an example, in many cases, a pesticide, such as 2,4-D, is made more water-soluble by converting it to its dimethylamine salt.

Molecules of Formula One may be formulated into stable complexes with a solvent, such that the complex remains intact after the non-complexed solvent is removed. These complexes are often referred to as “solvates.” However, it is particularly desirable to form stable hydrates with water as the solvent.

Molecules of Formula One may be made as various crystal polymorphs. Polymorphism is important in the development of agrochemicals since different crystal polymorphs or structures of the same molecule can have vastly different physical properties and biological performances.

Molecules of Formula One may be made with different isotopes. Of particular importance are molecules having ²H (also known as deuterium) in place of ¹H.

Molecules of Formula One may be made with different radionuclides. Of particular importance are molecules having ¹⁴C.

### Stereoisomers

Molecules of Formula One may exist as one or more stereoisomers. Thus, certain molecules can be produced as racemic mixtures. It will be appreciated by those skilled in the art that one stereoisomer may be more active than the other stereoisomers. Individual stereoisomers may be obtained by known selective synthetic procedures, by conventional synthetic procedures using resolved starting materials, or by conventional resolution procedures.

### Insecticides

Molecules of Formula One may also be used in combination (such as, in a compositional mixture, or a simultaneous or sequential application) with one or more of the following insecticides—1,2-dichloropropane, abamectin, acephate, acetamiprid, acethion, acetoprole, acrinathrin, acrylonitrile, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, beta-cyfluthrin, beta-cypermethrin, bifenthrin, bioallethrin, bioethanomethrin, biopermethrin, bistrifluoron, borax, boric acid, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclethrin, cycloprothrin, cyfluthrin, cyhalothrin, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, esdepallethrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, flubendiamide (additionally resolved isomers thereof), flucofuron, flucycloxuron, flucythrinate, flufenerim, flufenoxuron, flufenprox, fluvalinate, fonofos, formetanate, formetanate hydrochloride, formothion, formparanate, formparanate hydrochloride, fosmethilan, fospirate, fosthietan, fufenozide, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isodrin, isofenphos, isofenphos-methyl, isoprocarb, isoprothiolane, isothioate, isoxathion, ivermectin, jasmolin I, jasmolin II, jodfenphos, juvenile hormone I, juvenile hormone II, juvenile hormone III, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, meperfluthrin, mephosfolan, mercurous chloride, mesulfenfos, metaflumizone, methacrifos, methamidophos, methidathion, methiocarb, methocrotophos, methomyl, methoprene, methothrin, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, monocrotophos, monomehypo, monosultap, morphothion, moxidectin, naftalofos, naled, naphthalene, nicotine, nifluridide, nitenpyram, nithiazine, nitrilacarb, novaluron, noviflumuron, omethoate, oxamyl, oxydemeton-methyl, oxydeprofos, oxydisulfoton, para-dichlorobenzene, parathion, parathion-methyl, penfluoron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluralin, profluthrin, promacyl, promecarb, propaphos, propetamphos, propoxur, prothidathion, prothiofos, prothoate, protrifenbute, pymetrozine, pyraclofos, pyrafluprole, pyrazophos, pyresmethrin, pyrethrin I, pyrethrin II, pyrethrins, pyridaben, pyridalyl, pyridaphenthion, pyrifluquinazon, pyrimidifen, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetramethrin, tetramethylfluthrin, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tolfenpyrad, tralomethrin, transfluthrin, transpermethrin, triarathene, triazamate, triazophos, trichlorfon, trichlormetaphos-3, trichloronat, trifenofos, triflumuron, trimethacarb, triprene, vamidothion, vaniliprole, XMC, xylylcarb, zeta-cypermethrin, and zolaprofos (collectively these commonly named insecticides are defined as the “Insecticide Group”).

### Acaricides

Molecules of Formula One may also be used in combination (such as, in a compositional mixture, or a simultaneous or sequential application) with one or more of the following acaricides—acequinocyl, amidoflumet, arsenous oxide, azobenzene, azocyclotin, benomyl, benoxafos, benzoximate, benzyl benzoate, bifenazate, binapacryl, bromopropylate, chinomethionat, chlorbenside, chlorfenethol, chlorfenson, chlorfensulphide, chlorobenzilate, chloromebuform, chloromethiuron, chloropropylate, clofentezine, cyenopyrafen, cyflumetofen, cyhexatin, dichlofluanid, dicofol, dienochlor, diflovidazin, dinobuton, dinocap, dinocap-4, dinocap-6, dinocton, dinopenton, dinosulfon, dinoterbon, diphenyl sulfone, disulfuram, dofenapyn, etoxazole, fenazaquin, fenbutatin oxide, fenothiocarb, fenpyroximate, fenson, fentrifanil, fluacrypyrim, fluazuron, flubenzimine, fluenetil, flumethrin, fluorbenside, hexythiazox, mesulfen, MNAF, nikkomycins, proclonol, propargite, quintiofos, spirodiclofen, sulfuram, sulfur, tetradifon, tetranactin, tetrasul, and thioquinox (collectively these commonly named acaricides are defined as the “Acaricide Group”).

### Nematicides

Molecules of Formula One may also be used in combination (such as, in a compositional mixture, or a simultaneous or sequential application) with one or more of the following nematicides—1,3-dichloropropene, benclothiaz, dazomet, dazomet-sodium, DBCP, DCIP, diamidafos, fluensulfone, fosthiazate, furfural, imicyafos, isamidofos, isazofos, metam, metam-ammonium, metam-potassium, metam-sodium, phosphocarb, and thionazin (collectively these commonly named nematicides are defined as the “Nematicide Group”)

### Fungicides

Molecules of Formula One may also be used in combination (such as, in a compositional mixture, or a simultaneous or sequential application) with one or more of the following fungicides—(3-ethoxypropyl)mercury bromide, 2-methoxyethylmercury chloride, 2-phenylphenol, 8-hydroxyquinoline sulfate, 8-phenylmercurioxyquinoline, acibenzolar, acibenzolar-S-methyl, acypetacs, acypetacs-copper, acypetacs-zinc, aldimorph, allyl alcohol, ametoctradin, amisulbrom, ampropylfos, anilazine, aureofungin, azaconazole, azithiram, azoxystrobin, barium polysulfide, benalaxyl, benalaxyl-M, benodanil, benomyl, benquinox, bentaluron, benthiavalicarb, benthiavalicarb-isopropyl, benzalkonium chloride, benzamacril, benzamacril-isobutyl, benzamorf, benzohydroxamic acid, bethoxazin, binapacryl, biphenyl, bitertanol, bithionol, bixafen, blasticidin-S, Bordeaux mixture, boscalid, bromuconazole, bupirimate, Burgundy mixture, buthiobate, butylamine, calcium polysulfide, captafol, captan, carbamorph, carbendazim, carboxin, carpropamid, carvone, Cheshunt mixture, chinomethionat, chlobenthiazone, chloraniformethan, chloranil, chlorfenazole, chlorodinitronaphthalene, chloroneb, chloropicrin, chlorothalonil, chlorquinox, chlozolinate, climbazole, clotrimazole, copper acetate, copper carbonate, basic, copper hydroxide, copper naphthenate, copper oleate, copper oxychloride, copper silicate, copper sulfate, copper zinc chromate, cresol, cufraneb, cuprobam, cuprous oxide, cyazofamid, cyclafuramid, cycloheximide, cyflufenamid, cymoxanil, cypendazole, cyproconazole, cyprodinil, dazomet, dazomet-sodium, DBCP, debacarb, decafentin, dehydroacetic acid, dichlofluanid, dichlone, dichlorophen, dichlozoline, diclobutrazol, diclocymet, diclomezine, diclomezine-sodium, dicloran, diethofencarb, diethyl pyrocarbonate, difenoconazole, diflumetorim, dimethirimol, dimethomorph, dimoxystrobin, diniconazole, diniconazole-M, dinobuton, dinocap, dinocap-4, dinocap-6, dinocton, dinopenton, dinosulfon, dinoterbon, diphenylamine, dipyrithione, disulfuram, ditalimfos, dithianon, DNOC, DNOC-ammonium, DNOC-potassium, DNOC-sodium, dodemorph, dodemorph acetate, dodemorph benzoate, dodicin, dodicin-sodium, dodine, drazoxolon, edifenphos, epoxiconazole, etaconazole, etem, ethaboxam, ethirimol, ethoxyquin, ethylmercury 2,3-dihydroxypropyl mercaptide, ethylmercury acetate, ethylmercury bromide, ethylmercury chloride, ethylmercury phosphate, etridiazole, famoxadone, fenamidone, fenaminosulf, fenapanil, fenarimol, fenbuconazole, fenfuram, fenhexamid, fenitropan, fenoxanil, fenpiclonil, fenpropidin, fenpropimorph, fentin, fentin chloride, fentin hydroxide, ferbam, ferimzone, fluazinam, fludioxonil, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, furcarbanil, furconazole, furconazole-cis, furfural, furmecyclox, furophanate, glyodin, griseofulvin, guazatine, halacrinate, hexachlorobenzene, hexachlorobutadiene, hexaconazole, hexylthiofos, hydrargaphen, hymexazol, imazalil, imazalil nitrate, imazalil sulfate, imibenconazole, iminoctadine, iminoctadine triacetate, iminoctadine trialbesilate, iodomethane, ipconazole, iprobenfos, iprodione, iprovalicarb, isoprothiolane, isopyrazam, isotianil, isovaledione, kasugamycin, kresoxim-methyl, mancopper, mancozeb, mandipropamid, maneb, mebenil, mecarbinzid, mepanipyrim, mepronil, meptyldinocap, mercuric chloride, mercuric oxide, mercurous chloride, metalaxyl, metalaxyl-M, metam, metam-ammonium, metam-potassium, metam-sodium, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metominostrobin, metrafenone, metsulfovax, milneb, myclobutanil, myclozolin, N-(ethylmercury)-p-toluenesulphonanilide, nabam, natamycin, nitrostyrene, nitrothal-isopropyl, nuarimol, OCH, octhilinone, ofurace, orysastrobin, oxadixyl, oxine-copper, oxpoconazole, oxpoconazole fumarate, oxycarboxin, pefurazoate, penconazole, pencycuron, penflufen, pentachlorophenol, penthiopyrad, phenylmercuriurea, phenylmercury acetate, phenylmercury chloride, phenylmercury derivative of pyrocatechol, phenylmercury nitrate, phenylmercury salicylate, phosdiphen, phthalide, picoxystrobin, piperalin, polycarbamate, polyoxins, polyoxorim, polyoxorim-zinc, potassium azide, potassium polysulfide, potassium thiocyanate, probenazole, prochloraz, procymidone, propamocarb, propamocarb hydrochloride, propiconazole, propineb, proquinazid, prothiocarb, prothiocarb hydrochloride, prothioconazole, pyracarbolid, pyraclostrobin, pyraclostrobin, pyrametostrobin, pyraoxystrobin, pyrazophos, pyribencarb, pyridinitril, pyrifenox, pyrimethanil, pyriofenone, pyroquilon, pyroxychlor, pyroxyfur, quinacetol, quinacetol sulfate, quinazamid, quinconazole, quinoxyfen, quintozene, rabenzazole, salicylanilide, sedaxane, silthiofam, simeconazole, sodium azide, sodium orthophenylphenoxide, sodium pentachlorophenoxide, sodium polysulfide, spiroxamine, streptomycin, sulfur, sultropen, TCMTB, tebuconazole, tebufloquin, tecloftalam, tecnazene, tecoram, tetraconazole, thiabendazole, thiadifluor, thicyofen, thifluzamide, thiochlorfenphim, thiomersal, thiophanate, thiophanate-methyl, thioquinox, thiram, tiadinil, tioxymid, tolclofos-methyl, tolylfluanid, tolylmercury acetate, triadimefon, triadimenol, triamiphos, triarimol, triazbutil, triazoxide, tributyltin oxide, trichlamide, tricyclazole, tridemorph, trifloxystrobin, triflumizole, triforine, triticonazole, uniconazole, uniconazole-P, validamycin, valifenalate, vinclozolin, zarilamid, zinc naphthenate, zineb, ziram, zoxamide (collectively these commonly named fungicides are defined as the “Fungicide Group”).

### Herbicides

Molecules of Formula One may also be used in combination (such as, in a compositional mixture, or a simultaneous or sequential application) with one or more of the following herbicides—2,3,6-TBA, 2,3,6-TBA-dimethylammonium, 2,3,6-TBA-sodium, 2,4,5-T, 2,4,5-T-2-butoxypropyl, 2,4,5-T-2-ethylhexyl, 2,4,5-T-3-butoxypropyl, 2,4,5-TB, 2,4,5-T-butomethyl, 2,4,5-T-butotyl, 2,4,5-T-butyl, 2,4,5-T-isobutyl, 2,4,5-T-isoctyl, 2,4,5-T-isopropyl, 2,4,5-T-methyl, 2,4,5-T-pentyl, 2,4,5-T-sodium, 2,4,5-T-triethylammonium, 2,4,5-T-trolamine, 2,4-D, 2,4-D-2-butoxypropyl, 2,4-D-2-ethylhexyl, 2,4-D-3-butoxypropyl, 2,4-D-ammonium, 2,4-DB, 2,4-DB-butyl, 2,4-DB-dimethylammonium, 2,4-DB-isoctyl, 2,4-DB-potassium, 2,4-DB-sodium, 2,4-D-butotyl, 2,4-D-butyl, 2,4-D-diethylammonium, 2,4-D-dimethylammonium, 2,4-D-diolamine, 2,4-D-dodecylammonium, 2,4-DEB, 2,4-DEP, 2,4-D-ethyl, 2,4-D-heptylammonium, 2,4-D-isobutyl, 2,4-D-isoctyl, 2,4-D-isopropyl, 2,4-D-isopropylammonium, 2,4-D-lithium, 2,4-D-meptyl, 2,4-D-methyl, 2,4-D-octyl, 2,4-D-pentyl, 2,4-D-potassium, 2,4-D-propyl, 2,4-D-sodium, 2,4-D-tefuryl, 2,4-D-tetradecylammonium, 2,4-D-triethylammonium, 2,4-D-tris(2-hydroxypropyl)ammonium, 2,4-D-trolamine, 3,4-DA, 3,4-DB, 3,4-DP, 4-CPA, 4-CPB, 4-CPP, acetochlor, acifluorfen, acifluorfen-methyl, acifluorfen-sodium, aclonifen, acrolein, alachlor, allidochlor, alloxydim, alloxydim-sodium, allyl alcohol, alorac, ametridione, ametryn, amibuzin, amicarbazone, amidosulfuron, aminocyclopyrachlor, aminocyclopyrachlor-methyl, aminocyclopyrachlor-potassium, aminopyralid, aminopyralid-potassium, aminopyralid-tris(2-hydroxypropyl)ammonium, amiprofos-methyl, amitrole, ammonium sulfamate, anilofos, anisuron, asulam, asulam-potassium, asulam-sodium, atraton, atrazine, azafenidin, azimsulfuron, aziprotryne, barban, BCPC, beflubutamid, benazolin, benazolin-dimethylammonium, benazolin-ethyl, benazolin-potassium, bencarbazone, benfluralin, benfuresate, bensulfuron, bensulfuron-methyl, bensulide, bentazone, bentazone-sodium, benzadox, benzadox-ammonium, benzfendizone, benzipram, benzobicyclon, benzofenap, benzofluor, benzoylprop, benzoylprop-ethyl, benzthiazuron, bicyclopyrone, bifenox, bilanafos, bilanafos-sodium, bispyribac, bispyribac-sodium, borax, bromacil, bromacil-lithium, bromacil-sodium, bromobonil, bromobutide, bromofenoxim, bromoxynil, bromoxynil butyrate, bromoxynil heptanoate, bromoxynil octanoate, bromoxynil-potassium, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole, carfentrazone, carfentrazone-ethyl, CDEA, CEPC, chlomethoxyfen, chloramben, chloramben-ammonium, chloramben-diolamine, chloramben-methyl, chloramben-methylammonium, chloramben-sodium, chloranocryl, chlorazifop, chlorazifop-propargyl, chlorazine, chlorbromuron, chlorbufam, chloreturon, chlorfenac, chlorfenac-sodium, chlorfenprop, chlorfenprop-methyl, chlorflurazole, chlorflurenol, chlorflurenol-methyl, chloridazon, chlorimuron, chlorimuron-ethyl, chlornitrofen, chloropon, chlorotoluron, chloroxuron, chloroxynil, chlorprocarb, chlorpropham, chlorsulfuron, chlorthal, chlorthal-dimethyl, chlorthal-monomethyl, chlorthiamid, cinidon-ethyl, cinmethylin, cinosulfuron, cisanilide, clethodim, cliodinate, clodinafop, clodinafop-propargyl, clofop, clofop-isobutyl, clomazone, clomeprop, cloprop, cloproxydim, clopyralid, clopyralid-methyl, clopyralid-olamine, clopyralid-potassium, clopyralid-tris(2-hydroxypropyl)ammonium, cloransulam, cloransulam-methyl, CMA, copper sulfate, CPMF, CPPC, credazine, cresol, cumyluron, cyanamide, cyanatryn, cyanazine, cycloate, cyclosulfamuron, cycloxydim, cycluron, cyhalofop, cyhalofop-butyl, cyperquat, cyperquat chloride, cyprazine, cyprazole, cypromid, daimuron, dalapon, dalapon-calcium, dalapon-magnesium, dalapon-sodium, dazomet, dazomet-sodium, delachlor, desmedipham, desmetryn, di-allate, dicamba, dicamba-dimethylammonium, dicamba-diolamine, dicamba-isopropylammonium, dicamba-methyl, dicamba-olamine, dicamba-potassium, dicamba-sodium, dicamba-trolamine, dichlobenil, dichloralurea, dichlormate, dichlorprop, dichlorprop-2-ethylhexyl, dichlorprop-butotyl, dichlorprop-dimethylammonium, dichlorprop-ethylammonium, dichlorprop-isoctyl, dichlorprop-methyl, dichlorprop-P, dichlorprop-P-dimethylammonium, dichlorprop-potassium, dichlorprop-sodium, diclofop, diclofop-methyl, diclosulam, diethamquat, diethamquat dichloride, diethatyl, diethatyl-ethyl, difenopenten, difenopenten-ethyl, difenoxuron, difenzoquat, difenzoquat metilsulfate, diflufenican, diflufenzopyr, diflufenzopyr-sodium, dimefuron, dimepiperate, dimethachlor, dimethametryn, dimethenamid, dimethenamid-P, dimexano, dimidazon, dinitramine, dinofenate, dinoprop, dinosam, dinoseb, dinoseb acetate, dinoseb-ammonium, dinoseb-diolamine, dinoseb-sodium, dinoseb-trolamine, dinoterb, dinoterb acetate, diphacinone-sodium, diphenamid, dipropetryn, diquat, diquat dibromide, disul, disul-sodium, dithiopyr, diuron, DMPA, DNOC, DNOC-ammonium, DNOC-potassium, DNOC-sodium, DSMA, EBEP, eglinazine, eglinazine-ethyl, endothal, endothal-diammonium, endothal-dipotassium, endothal-disodium, epronaz, EPTC, erbon, esprocarb, ethalfluralin, ethametsulfuron, ethametsulfuron-methyl, ethidimuron, ethiolate, ethofumesate, ethoxyfen, ethoxyfen-ethyl, ethoxysulfuron, etinofen, etnipromid, etobenzanid, EXD, fenasulam, fenoprop, fenoprop-3-butoxypropyl, fenoprop-butomethyl, fenoprop-butotyl, fenoprop-butyl, fenoprop-isoctyl, fenoprop-methyl, fenoprop-potassium, fenoxaprop, fenoxaprop-ethyl, fenoxaprop-P, fenoxaprop-P-ethyl, fenoxasulfone, fenteracol, fenthiaprop, fenthiaprop-ethyl, fentrazamide, fenuron, fenuron TCA, ferrous sulfate, flamprop, flamprop-isopropyl, flamprop-M, flamprop-methyl, flamprop-M-isopropyl, flamprop-M-methyl, flazasulfuron, florasulam, fluazifop, fluazifop-butyl, fluazifop-methyl, fluazifop-P, fluazifop-P-butyl, fluazolate, flucarbazone, flucarbazone-sodium, flucetosulfuron, fluchloralin, flufenacet, flufenican, flufenpyr, flufenpyr-ethyl, flumetsulam, flumezin, flumiclorac, flumiclorac-pentyl, flumioxazin, flumipropyn, fluometuron, fluorodifen, fluoroglycofen, fluoroglycofen-ethyl, fluoromidine, fluoronitrofen, fluothiuron, flupoxam, flupropacil, flupropanate, flupropanate-sodium, flupyrsulfuron, flupyrsulfuron-methyl-sodium, fluridone, fluorochloridone, fluoroxypyr, fluoroxypyr-butomethyl, fluoroxypyr-meptyl, flurtamone, fluthiacet, fluthiacet-methyl, fomesafen, fomesafen-sodium, foramsulfuron, fosamine, fosamine-ammonium, furyloxyfen, glufosinate, glufosinate-ammonium, glufosinate-P, glufosinate-P-ammonium, glufosinate-P-sodium, glyphosate, glyphosate-diammonium, glyphosate-dimethylammonium, glyphosate-isopropylammonium, glyphosate-monoammonium, glyphosate-potassium, glyphosate-sesquisodium, glyphosate-trimesium, halosafen, halosulfuron, halosulfuron-methyl, haloxydine, haloxyfop, haloxyfop-etotyl, haloxyfop-methyl, haloxyfop-P, haloxyfop-P-etotyl, haloxyfop-P-methyl, haloxyfop-sodium, hexachloroacetone, hexaflurate, hexazinone, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazaquin-methyl, imazaquin-sodium, imazethapyr, imazethapyr-ammonium, imazosulfuron, indanofan, indaziflam, iodobonil, iodomethane, iodosulfuron, iodosulfuron-methyl-sodium, ioxynil, ioxynil octanoate, ioxynil-lithium, ioxynil-sodium, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lactofen, lenacil, linuron, MAA, MAMA, MCPA, MCPA-2-ethylhexyl, MCPA-butotyl, MCPA-butyl, MCPA-dimethylammonium, MCPA-diolamine, MCPA-ethyl, MCPA-isobutyl, MCPA-isoctyl, MCPA-isopropyl, MCPA-methyl, MCPA-olamine, MCPA-potassium, MCPA-sodium, MCPA-thioethyl, MCPA-trolamine, MCPB, MCPB-ethyl, MCPB-methyl, MCPB-sodium, mecoprop, mecoprop-2-ethylhexyl, mecoprop-dimethylammonium, mecoprop-diolamine, mecoprop-ethadyl, mecoprop-isoctyl, mecoprop-methyl, mecoprop-P, mecoprop-P-dimethylammonium, mecoprop-P-isobutyl, mecoprop-potassium, mecoprop-P-potassium, mecoprop-sodium, mecoprop-trolamine, medinoterb, medinoterb acetate, mefenacet, mefluidide, mefluidide-diolamine, mefluidide-potassium, mesoprazine, mesosulfuron, mesosulfuron-methyl, mesotrione, metam, metam-ammonium, metamifop, metamitron, metam-potassium, metam-sodium, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metolachlor, metosulam, metoxuron, metribuzin, metsulfuron, metsulfuron-methyl, molinate, monalide, monisouron, monochloroacetic acid, monolinuron, monuron, monuron TCA, morfamquat, morfamquat dichloride, MSMA, naproanilide, napropamide, naptalam, naptalam-sodium, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxadiargyl, oxadiazon, oxapyrazon, oxapyrazon-dimolamine, oxapyrazon-sodium, oxasulfuron, oxaziclomefone, oxyfluorfen, parafluoron, paraquat, paraquat dichloride, paraquat dimetilsulfate, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picloram-2-ethylhexyl, picloram-isoctyl, picloram-methyl, picloram-olamine, picloram-potassium, picloram-triethylammonium, picloram-tris(2-hydroxypropyl)ammonium, picolinafen, pinoxaden, piperophos, potassium arsenite, potassium azide, potassium cyanate, pretilachlor, primisulfuron, primisulfuron-methyl, procyazine, prodiamine, profluazol, profluralin, profoxydim, proglinazine, proglinazine-ethyl, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, proxan, proxan-sodium, prynachlor, pydanon, pyraclonil, pyraflufen, pyraflufen-ethyl, pyrasulfotole, pyrazolynate, pyrazosulfuron, pyrazosulfuron-ethyl, pyrazoxyfen, pyribenzoxim, pyributicarb, pyriclor, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quinonamid, quizalofop, quizalofop-ethyl, quizalofop-P, quizalofop-P-ethyl, quizalofop-P-tefuryl, rhodethanil, rimsulfuron, saflufenacil, sebuthylazine, secbumeton, sethoxydim, siduron, simazine, simeton, simetryn, SMA, S-metolachlor, sodium arsenite, sodium azide, sodium chlorate, sulcotrione, sulfallate, sulfentrazone, sulfometuron, sulfometuron-methyl, sulfosulfuron, sulfuric acid, sulglycapin, swep, TCA, TCA-ammonium, TCA-calcium, TCA-ethadyl, TCA-magnesium, TCA-sodium, tebutam, tebuthiuron, tefuryltrione, tembotrione, tepraloxydim, terbacil, terbucarb, terbuchlor, terbumeton, terbuthylazine, terbutryn, tetrafluoron, thenylchlor, thiazafluoron, thiazopyr, thidiazimin, thidiazuron, thiencarbazone, thiencarbazone-methyl, thifensulfuron, thifensulfuron-methyl, thiobencarb, tiocarbazil, tioclorim, topramezone, tralkoxydim, tri-allate, triasulfuron, triaziflam, tribenuron, tribenuron-methyl, tricamba, triclopyr, triclopyr-butotyl, triclopyr-ethyl, triclopyr-triethylammonium, tridiphane, trietazine, trifloxysulfuron, trifloxysulfuron-sodium, trifluralin, triflusulfuron, triflusulfuron-methyl, trifop, trifop-methyl, trifopsime, trihydroxytriazine, trimeturon, tripropindan, tritac, tritosulfuron, vernolate, xylachlor, (collectively these commonly named herbicides are defined as the “Herbicide Group”).

### Biopesticides

Molecules of Formula One may also be used in combination (such as in a compositional mixture, or a simultaneous or sequential application) with one or more biopesticides. The term “biopesticide” is used for microbial biological pest control agents that are applied in a similar manner to chemical pesticides. Commonly these are bacterial, but there are also examples of fungal control agents, including Trichoderma spp. and Ampelomyces quisqualis (a control agent for grape powdery mildew). Bacillus subtilis are used to control plant pathogens. Weeds and rodents have also been controlled with microbial agents. One well-known insecticide example is Bacillus thuringiensis, a bacterial disease of Lepidoptera, Coleoptera, and Diptera. Because it has little effect on other organisms, it is considered more environmentally friendly than synthetic pesticides. Biological insecticides include products based on:

1. entomopathogenic fungi (e.g. Metarhizium anisopliae);

2. entomopathogenic nematodes (e.g. Steinemema feltiae); and

3. entomopathogenic viruses (e.g. Cydia pomonella granulovirus).

Other examples of entomopathogenic organisms include, but are not limited to, baculoviruses, bacteria and other prokaryotic organisms, fungi, protozoa and Microsproridia. Biologically derived insecticides include, but not limited to, rotenone, veratridine, as well as microbial toxins; insect tolerant or resistant plant varieties; and organisms modified by recombinant DNA technology to either produce insecticides or to convey an insect resistant property to the genetically modified organism. In one embodiment, the molecules of Formula One may be used with one or more biopesticides in the area of seed treatments and soil amendments. The Manual of Biocontrol Agents gives a review of the available biological insecticide (and other biology-based control) products. Copping L. G. (ed.) (2004). The Manual of Biocontrol Agents (formerly the Biopesticide Manual) 3rd Edition. British Crop Production Council (BCPC), Farnham, Surrey UK.

### Other Active Compounds

Molecules of Formula One may also be used in combination (such as in a compositional mixture, or a simultaneous or sequential application) with one or more of the following:
1. 3-(4-chloro-2,6-dimethylphenyl)-4-hydroxy-8-oxa-1-azaspiro[4,5]dec-3-en-2-one;
2. 3-(4′-chloro-2,4-dimethyl[1,1′-biphenyl]-3-yl)-4-hydroxy-8-oxa-1-azaspiro[4,5]dec-3-en-2-one;
3. 4-[[(6-chloro-3-pyridinyl)methyl]methylamino]-2(5H)-furanone;
4. 4-[[(6-chloro-3-pyridinyl)methyl]cyclopropylamino]-2(5H)-furanone;
5. 3-chloro-N2-[(1S)-1-methyl-2-(methylsulfonyl)ethyl]-N1-[2-methyl-4-[1,2,2,2-tetrafluoro-1-(trifluoromethyl)ethyl]phenyl]-1,2-benzenedicarboxamide;
6. 2-cyano-N-ethyl-4-fluoro-3-methoxy-benzenesulfonamide;
7. 2-cyano-N-ethyl-3-methoxy-benzenesulfonamide;
8. 2-cyano-3-difluoromethoxy-N-ethyl-4-fluoro-benzenesulfonamide;
9. 2-cyano-3-fluoromethoxy-N-ethyl-benzenesulfonamide;
10. 2-cyano-6-fluoro-3-methoxy-N,N-dimethyl-benzenesulfonamide;
11. 2-cyano-N-ethyl-6-fluoro-3-methoxy-N-methyl-benzenesulfonamide;
12. 2-cyano-3-difluoromethoxy-N,N-dimethylbenzenesulfon-amide;
13. 3-(difluoromethyl)-N-[2-(3,3-dimethylbutyl)phenyl]-1-methyl-1H-pyrazole-4-carboxamide;
14. N-ethyl-2,2-dimethylpropionamide-2-(2,6-dichloro-α,α,α-trifluoro-p-tolyl)hydrazone;
15. N-ethyl-2,2-dichloro-1-methylcyclopropane-carboxamide-2-(2,6-dichloro-α,α,α-trifluoro-p-tolyl)hydrazone nicotine;
16. O-{(E-)-[2-(4-chloro-phenyl)-2-cyano-1-(2-trifluoromethylphenyl)-vinyl]}S-methyl thiocarbonate;
17. (E)-N1-[(2-chloro-1,3-thiazol-5-ylmethyl)]-N2-cyano-N1-methylacetamidine;
18. 1-(6-chloropyridin-3-ylmethyl)-7-methyl-8-nitro-1,2,3,5,6,7-hexahydro-imidazo[1,2-a]pyridin-5-ol;
19. 4-[4-chlorophenyl-(2-butylidine-hydrazono)methyl)]phenyl mesylate; and
20. N-Ethyl-2,2-dichloro-1-methylcyclopropanecarboxamide-2-(2,6-dichloro-alpha,alpha,alpha-trifluoro-p-tolyl)hydrazone.

Molecules of Formula One may also be used in combination (such as in a compositional mixture, or a simultaneous or sequential application) with one or more compounds in the following groups: algicides, antifeedants, avicides, bactericides, bird repellents, chemosterilants, herbicide safeners, insect attractants, insect repellents, mammal repellents, mating disrupters, molluscicides, plant activators, plant growth regulators, rodenticides, and/or virucides (collectively these commonly named groups are defined as the “AI Group”). It should be noted that compounds falling within the AI Group, Insecticide Group, Fungicide Group, Herbicide Group, Acaricide Group, or Nematicide Group might be in more than one group, because of multiple activities the compound has. For more information consult the “COMPENDIUM OF PESTICIDE COMMON NAMES” located at http://www.alanwood.net/pesticides/index.html. Also consult “THE PESTICIDE MANUAL” 14th Edition, edited by C D S Tomlin, copyright 2006 by British Crop Production Council, or its prior or more recent editions.

### Synergistic Mixtures and Synergists

Molecules of Formula One may be used with the compounds in the Insecticide Group to form synergistic mixtures where the mode of action of such compounds compared to the mode of action of the molecules of Formula One are the same, similar, or different. Examples of modes of action include, but are not limited to: acetylcholinesterase inhibitor; sodium channel modulator; chitin biosynthesis inhibitor; GABA-gated chloride channel antagonist; GABA and glutamate-gated chloride channel agonist; acetylcholine receptor agonist; MET I inhibitor; Mg-stimulated ATPase inhibitor; nicotinic acetylcholine receptor; Midgut membrane disrupter; oxidative phosphorylation disrupter, and ryanodine receptor (RyRs). Additionally, molecules of Formula One may be used with compounds in the Fungicide Group, Acaricide Group, Herbicide Group, or Nematicide Group to form synergistic mixtures. Furthermore, molecules of Formula One may be used with other active compounds, such as the compounds under the heading “OTHER ACTIVE COMPOUNDS”, algicides, avicides, bactericides, molluscicides, rodenticides, virucides, herbicide safeners, adjuvants, and/or surfactants to form synergistic mixtures. Generally, weight ratios of the molecules of Formula One in a synergistic mixture with another compound are from about 10:1 to about 1:10, preferably from about 5:1 to about 1:5, and more preferably from about 3:1, and even more preferably about 1:1. Additionally, the following compounds are known as synergists and may be used with the molecules disclosed in Formula One: piperonyl butoxide, piprotal, propyl isome, sesamex, sesamolin, sulfoxide, and tribufos (collectively these synergists are defined as the “Synergists Group”).

### Formulations

A pesticide is rarely suitable for application in its pure form. It is usually necessary to add other substances so that the pesticide can be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water-soluble liquids, water-dispersible granules or dry flowables, wettable powders, and ultra low volume solutions. For further information on formulation types see “Catalogue of Pesticide Formulation Types and International Coding System” Technical Monograph no 2, 5th Edition by CropLife International (2002).

Pesticides are applied most often as aqueous suspensions or emulsions prepared from concentrated formulations of such pesticides. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually chosen from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier. It is often most effective to grind and mix the pesticide at the same time by preparing the aqueous mixture and homogenizing it in an implement such as a sand mill, ball mill, or piston-type homogenizer.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. They can be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait they also consume the pesticide. Baits may take the form of granules, gels, flowable powders, liquids, or solids. They can be used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings or in special chambers.

Pesticides can be microencapsulated by suspending the pesticide particles or droplets in plastic polymers of various types. By altering the chemistry of the polymer or by changing factors in the processing, microcapsules can be formed of various sizes, solubility, wall thicknesses, and degrees of penetrability. These factors govern the speed with which the active ingredient within is released, which in turn, affects the residual performance, speed of action, and odor of the product.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer comprising: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007, having patent application Ser. No. 11/495,228. For ease of use, this embodiment will be referred to as “OIWE”.

For further information consult “Insect Pest Management” 2nd Edition by D. Dent, copyright CAB International (2000). Additionally, for more detailed information consult “Handbook of Pest Control—The Behavior, Life History, and Control of Household Pests” by Arnold Mattis, 9th Edition, copyright 2004 by GIE Media Inc.

### Other Formulation Components

Generally, when the molecules disclosed in Formula One are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g. plant and animal sources. Specific examples are: vegetable oils, seed oils, and esters thereof, also alkoxylated alkyl polyglucosides.

For further information, see “Chemistry and Technology of Agrochemical Formulations” edited by D. A. Knowles, copyright 1998 by Kluwer Academic Publishers. Also see “Insecticides in Agriculture and Environment—Retrospects and Prospects” by A. S. Perry, I. Yamamoto, I. Ishaaya, and R. Perry, copyright 1998 by Springer-Verlag.

### Pests

In general, the molecules of Formula One may be used to control pests e.g. beetles, earwigs, cockroaches, flies. aphids, scales, whiteflies, leafhoppers, ants, wasps, termites, moths, butterflies, lice, grasshoppers, locusts, crickets, fleas, thrips, bristletails, mites, ticks, nematodes, and symphylans.

In another embodiment, the molecules of Formula One may be used to control pests in the Phyla Nematoda and/or Arthropoda.

In another embodiment, the molecules of Formula One may be used to control pests in the Subphyla Chelicerata, Myriapoda, and/or Hexapoda.

In another embodiment, the molecules of Formula One may be used to control pests in the Classes of Arachnida, Symphyla, and/or Insecta.

In another embodiment, the molecules of Formula One may be used to control pests of the Order Anoplura. A non-exhaustive list of particular genera includes, but is not limited to, Haematopinus spp., Hoplopleura spp., Linognathus spp., Pediculus spp., and Polyplax spp. A non-exhaustive list of particular species includes, but is not limited to, Haematopinus asini, Haematopinus suis, Linognathus setosus, Linognathus ovillus, Pediculus humanus capitis, Pediculus humanus humanus, and Pthirus pubis.

In another embodiment, the molecules of Formula One may be used to control pests in the Order Coleoptera. A non-exhaustive list of particular genera includes, but is not limited to, Acanthoscelides spp., Agriotes spp., Anthonomus spp., Apion spp., Apogonia spp., Aulacophora spp., Bruchus spp., Cerosterna spp., Cerotoma spp., Ceutorhynchus spp., Chaetocnema spp., Colaspis spp., Ctenicera spp., Curculio spp., Cyclocephala spp., Diabrotica spp., Hypera spp., Ips spp., Lyctus spp., Megascelis spp., Meligethes spp., Otiorhynchus spp., Pantomorus spp., Phyllophaga spp., Phyllotreta spp., Rhizotrogus spp., Rhynchites spp., Rhynchophorus spp., Scolytus spp., Sphenophorus spp., Sitophilus spp., and Tribolium spp. A non-exhaustive list of particular species includes, but is not limited to, Acanthoscelides obtectus, Agrilus planipennis, Anoplophora glabripennis, Anthonomus grandis, Ataenius spretulus, Atomaria linearis, Bothynoderes punctiventris, Bruchus pisorum, Callosobruchus maculatus, Carpophilus hemipterus, Cassida vittata, Cerotoma trifurcata, Ceutorhynchus assimilis, Ceutorhynchus napi, Conoderus scalaris, Conoderus stigmosus, Conotrachelus nenuphar, Cotinis nitida, Crioceris asparagi, Cryptolestes ferrugineus, Cryptolestes pusillus, Cryptolestes turcicus, Cylindrocopturus adspersus, Deporaus marginatus, Dermestes lardarius, Dermestes maculatus, Epilachna varivestis, Faustinus cubae, Hylobius pales, Hypera postica, Hypothenemus hampei, Lasioderma serricorne, Leptinotarsa decemlineata, Liogenys fuscus, Liogenys suturalis, Lissorhoptrus oryzophilus, Maecolaspis joliveti, Melanotus communis, Meligethes aeneus, Melolontha melolontha, Oberea brevis, Oberea linearis, Oryctes rhinoceros, Oryzaephilus mercator, Oryzaephilus surinamensis, Oulema melanopus, Oulema oryzae, Phyllophaga cuyabana, Popillia japonica, Prostephanus truncatus, Rhyzopertha dominica, Sitona lineatus, Sitophilus granarius, Sitophilus oryzae, Sitophilus zeamais, Stegobium paniceum, Tribolium castaneum, Tribolium confusum, Trogoderma variabile, and Zabrus tenebrioides.

In another embodiment, the molecules of Formula One may be used to control pests of the Order Dermaptera.

In another embodiment, the molecules of Formula One may be used to control pests of the Order Blattaria. A non-exhaustive list of particular species includes, but is not limited to, Blattella germanica, Blatta orientalis, Parcoblatta pennsylvanica, Periplaneta americana, Periplaneta australasiae, Periplaneta brunnea, Periplaneta fuliginosa, Pycnoscelus surinamensis, and Supella longipalpa.

In another embodiment, the molecules of Formula One may be used to control pests of the Order Diptera. A non-exhaustive list of particular genera includes, but is not limited to, Aedes spp., Agromyza spp., Anastrepha spp., Anopheles spp., Bactrocera spp., Ceratitis spp., Chrysops spp., Cochliomyia spp., Contarinia spp., Culex spp., Dasineura spp., Delia spp., Drosophila spp., Fannia spp., Hylemyia spp., Liriomyza spp., Musca spp., Phorbia spp., Tabanus spp., and Tipula spp. A non-exhaustive list of particular species includes, but is not limited to, Agromyza frontella, Anastrepha suspensa, Anastrepha ludens, Anastrepha obliqa, Bactrocera cucurbitae, Bactrocera dorsalis, Bactrocera invadens, Bactrocera zonata, Ceratitis capitata, Dasineura brassicae, Delia platura, Fannia canicularis, Fannia scalaris, Gasterophilus intestinalis, Gracillia perseae, Haematobia irritans, Hypoderma lineatum, Liriomyza brassicae, Melophagus ovinus, Musca autumnalis, Musca domestica, Oestrus ovis, Oscinella frit, Pegomya betae, Psila rosae, Rhagoletis cerasi, Rhagoletis pomonella, Rhagoletis mendax, Sitodiplosis mosellana, and Stomoxys calcitrans.

In another embodiment, the molecules of Formula One may be used to control pests of the Order Hemiptera. A non-exhaustive list of particular genera includes, but is not limited to, Adelges spp., Aulacaspis spp., Aphrophora spp., Aphis spp., Bemisia spp., Ceroplastes spp., Chionaspis spp., Chrysomphalus spp., Coccus spp., Empoasca spp., Lepidosaphes spp., Lagynotomus spp., Lygus spp., Macrosiphum spp., Nephotettix spp., Nezara spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp. and Unaspis spp. A non-exhaustive list of particular species includes, but is not limited to, Acrosternum hilare, Acyrthosiphon pisum, Aleyrodes proletella, Aleurodicus dispersus, Aleurothrixus floccosus, Amrasca biguttula biguttula, Aonidiella aurantii, Aphis gossypii, Aphis glycines, Aphis pomi, Aulacorthum solani, Bemisia argentifolii, Bemisia tabaci, Blissus leucopterus, Brachycorynella asparagi, Brevennia rehi, Brevicoryne brassicae, Calocoris norvegicus, Ceroplastes rubens, Cimex hemipterus, Cimex lectularius, Dagbertus fasciatus, Dichelops furcatus, Diuraphis noxia, Diaphorina citri, Dysaphis plantaginea, Dysdercus suturellus, Edessa meditabunda, Eriosoma lanigerum, Eurygaster maura, Euschistus heros, Euschistus servus, Helopeltis antonii, Helopeltis theivora, Icerya purchasi, Idioscopus nitidulus, Laodelphax striatellus, Leptocorisa oratorius, Leptocorisa varicornis, Lygus hesperus, Maconellicoccus hirsutus, Macrosiphum euphorbiae, Macrosiphum granarium, Macrosiphum rosae, Macrosteles quadrilineatus, Mahanarva frimbiolata, Metopolophium dirhodum, Mictis longicornis, Myzus persicae, Nephotettix cinctipes, Neurocolpus longirostris, Nezara viridula, Nilaparvata lugens, Parlatoria pergandii, Parlatoria ziziphi, Peregrinus maidis, Phylloxera vitifoliae, Physokermes piceae, Phytocoris californicus, Phytocoris relativus, Piezodorus guildinii, Poecilocapsus lineatus, Psallus vaccinicola, Pseudacysta perseae, Pseudococcus brevipes, Quadraspidiotus perniciosus, Rhopalosiphum maidis, Rhopalosiphum padi, Saissetia oleae, Scaptocoris castanea, Schizaphis graminum, Sitobion avenae, Sogatella furcifera, Trialeurodes vaporariorum, Trialeurodes abutiloneus, Unaspis yanonensis, and Zulia entrerriana.

In another embodiment, the molecules of Formula One may be used to control pests of the Order Hymenoptera. A non-exhaustive list of particular genera includes, but is not limited to, Acromyrmex spp., Atta spp., Camponotus spp., Diprion spp., Formica spp., Monomorium spp., Neodiprion spp., Pogonomyrmex spp., Polistes spp., Solenopsis spp., Vespula spp., and Xylocopa spp. A non-exhaustive list of particular species includes, but is not limited to, Athalia rosae, Atta texana, Iridomyrmex humilis, Monomorium minimum, Monomorium pharaonis, Solenopsis invicta, Solenopsis geminata, Solenopsis molesta, Solenopsis richtery, Solenopsis xyloni, and Tapinoma sessile.

In another embodiment, the molecules of Formula One may be used to control pests of the Order Isoptera. A non-exhaustive list of particular genera includes, but is not limited to, Coptotermes spp., Cornitermes spp., Cryptotermes spp., Heterotermes spp., Kalotermes spp., Incisitermes spp., Macrotermes spp., Marginitermes spp., Microcerotermes spp., Procornitermes spp., Reticulitermes spp., Schedorhinotermes spp., and Zootermopsis spp. A non-exhaustive list of particular species includes, but is not limited to, Coptotermes curvignathus, Coptotermes frenchi, Coptotermes formosanus, Heterotermes aureus, Microtermes obesi, Reticulitermes banyulensis, Reticulitermes grassei, Reticulitermes flavipes, Reticulitermes hageni, Reticulitermes hesperus, Reticulitermes santonensis, Reticulitermes speratus, Reticulitermes tibialis, and Reticulitermes virginicus.

In another embodiment, the molecules of Formula One may be used to control pests of the Order Lepidoptera. A non-exhaustive list of particular genera includes, but is not limited to, Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Peridroma spp., Phyllonorycter spp., Pseudaletia spp., Sesamia spp., Spodoptera spp., Synanthedon spp., and Yponomeuta spp. A non-exhaustive list of particular species includes, but is not limited to, Achaea janata, Adoxophyes orana, Agrotis ipsilon, Alabama argillacea, Amorbia cuneana, Amyelois transitella, Anacamptodes defectaria, Anarsia lineatella, Anomis sabulifera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Cossus cossus, Cydia caryana, Cydia funebrana, Cydia molesta, Cydia nigricana, Cydia pomonella, Darna diducta, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Eupoecilia ambiguella, Euxoa auxiliaris, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera malifoliella, Lobesia botrana, Loxagrotis albicosta, Lymantria dispar, Lyonetia clerkella, Mahasena corbetti, Mamestra brassicae, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Pieris rapae, Plathypena scabra, Plodia interpunctella, Plutella xylostella, Polychrosis viteana, Prays endocarpa, Prays oleae, Pseudaletia unipuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tineola bisselliella, Trichoplusia ni, Tuta absoluta, Zeuzera coffeae, and Zeuzera pyrina.

In another embodiment, the molecules of Formula One may be used to control pests of the Order Mallophaga. A non-exhaustive list of particular genera includes, but is not limited to, Anaticola spp., Bovicola spp., Chelopistes spp., Goniodes spp., Menacanthus spp., and Trichodectes spp. A non-exhaustive list of particular species includes, but is not limited to, Bovicola bovis, Bovicola caprae, Bovicola ovis, Chelopistes meleagridis, Goniodes dissimilis, Goniodes gigas, Menacanthus stramineus, Menopon gallinae, and Trichodectes canis.

In another embodiment, the molecules of Formula One may be used to control pests of the Order Orthoptera. A non-exhaustive list of particular genera includes, but is not limited to, Melanoplus spp., and Pterophylla spp. A non-exhaustive list of particular species includes, but is not limited to, Anabrus simplex, Gryllotalpa africana, Gryllotalpa australis, Gryllotalpa brachyptera, Gryllotalpa hexadactyla, Locusta migratoria, Microcentrum retinerve, Schistocerca gregaria, and Scudderia furcata.

In another embodiment, the molecules of Formula One may be used to control pests of the Order Siphonaptera. A non-exhaustive list of particular species includes, but is not limited to, Ceratophyllus gallinae, Ceratophyllus niger, Ctenocephalides canis, Ctenocephalides felis, and Pulex irritans.

In another embodiment, the molecules of Formula One may be used to control pests of the Order Thysanoptera. A non-exhaustive list of particular genera includes, but is not limited to, Caliothrips spp., Frankliniella spp., Scirtothrips spp., and Thrips spp. A non-exhaustive list of particular sp. includes, but is not limited to, Frankliniella fusca, Frankliniella occidentalis, Frankliniella schultzei, Frankliniella williamsi, Heliothrips haemorrhoidalis, Rhipiphorothrips cruentatus, Scirtothrips citri, Scirtothrips dorsalis, and Taeniothrips rhopalantennalis, Thrips hawaiiensis, Thrips nigropilosus, Thrips orientalis, Thrips tabaci.

In another embodiment, the molecules of Formula One may be used to control pests of the Order Thysanura. A non-exhaustive list of particular genera includes, but is not limited to, Lepisma spp. and Thermobia spp.

In another embodiment, the molecules of Formula One may be used to control pests of the Order Acarina. A non-exhaustive list of particular genera includes, but is not limited to, Acarus spp., Aculops spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. A non-exhaustive list of particular species includes, but is not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Notoedres cati, Oligonychus coffeae, Oligonychus ilicis, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, and Varroa destructor.

In another embodiment, the molecules of Formula One may be used to control pest of the Order Symphyla. A non-exhaustive list of particular sp. includes, but is not limited to, Scutigerella immaculata.

In another embodiment, the molecules of Formula One may be used to control pests of the Phylum Nematoda. A non-exhaustive list of particular genera includes, but is not limited to, Aphelenchoides spp., Belonolaimus spp., Criconemella spp., Ditylenchus spp., Heterodera spp., Hirschmanniella spp., Hoplolaimus spp., Meloidogyne spp., Pratylenchus spp., and Radopholus spp. A non-exhaustive list of particular sp. includes, but is not limited to, Dirofilaria immitis, Heterodera zeae, Meloidogyne incognita, Meloidogyne javanica, Onchocerca volvulus, Radopholus similis, and Rotylenchulus reniformis.

For additional information consult “HANDBOOK OF PEST CONTROL—THE BEHAVIOR, LIFE HISTORY, AND CONTROL OF HOUSEHOLD PESTS” by Arnold Mattis, 9th Edition, copyright 2004 by GIE Media Inc.

### Applications

Molecules of Formula One are generally used in amounts from about 0.01 grams per hectare to about 5000 grams per hectare to provide control. Amounts from about 0.1 grams per hectare to about 500 grams per hectare are generally preferred, and amounts from about 1 gram per hectare to about 50 grams per hectare are generally more preferred.

The area to which a molecule of Formula One is applied can be any area inhabited (or maybe inhabited, or traversed by) a pest, for example: where crops, trees, fruits, cereals, fodder species, vines, turf and ornamental plants, are growing; where domesticated animals are residing; the interior or exterior surfaces of buildings (such as places where grains are stored), the materials of construction used in building (such as impregnated wood), and the soil around buildings. Particular crop areas to use a molecule of Formula One include areas where apples, corn, sunflowers, cotton, soybeans, canola, wheat, rice, sorghum, barley, oats, potatoes, oranges, alfalfa, lettuce, strawberries, tomatoes, peppers, crucifers, pears, tobacco, almonds, sugar beets, beans and other valuable crops are growing or the seeds thereof are going to be planted. It is also advantageous to use aluminum sulfate with a molecule of Formula One when growing various plants.

Controlling pests generally means that pest populations, pest activity, or both, are reduced in an area. This can come about when: pest populations are repulsed from an area; when pests are incapacitated in or around an area; or pests are exterminated, in whole, or in part, in or around an area. Of course, a combination of these results can occur. Generally, pest populations, activity, or both are desirably reduced more than fifty percent, preferably more than 90 percent. Generally, the area is not in or on a human; consequently, the locus is generally a non-human area.

The molecules of Formula One may be used in mixtures, applied simultaneously or sequentially, alone or with other compounds to enhance plant vigor (e.g. to grow a better root system, to better withstand stressful growing conditions). Such other compounds are, for example, compounds that modulate plant ethylene receptors, most notably 1-methylcyclopropene (also known as 1-MCP).

The molecules of Formula One can be applied to the foliar and fruiting portions of plants to control pests. The molecules will either come in direct contact with the pest, or the pest will consume the pesticide when eating leaf, fruit mass, or extracting sap, that contains the pesticide. The molecules of Formula One can also be applied to the soil, and when applied in this manner, root and stem feeding pests can be controlled. The roots can absorb a molecule taking it up into the foliar portions of the plant to control above ground chewing and sap feeding pests.

Generally, with baits, the baits are placed in the ground where, for example, termites can come into contact with, and/or be attracted to, the bait. Baits can also be applied to a surface of a building, (horizontal, vertical, or slant surface) where, for example, ants, termites, cockroaches, and flies, can come into contact with, and/or be attracted to, the bait. Baits can comprise a molecule of Formula One.

The molecules of Formula One can be encapsulated inside, or placed on the surface of a capsule. The size of the capsules can range from nanometer size (about 100-900 nanometers in diameter) to micrometer size (about 10-900 microns in diameter).

Because of the unique ability of the eggs of some pests to resist certain pesticides, repeated applications of the molecules of Formula One may be desirable to control newly emerged larvae.

Systemic movement of pesticides in plants may be utilized to control pests on one portion of the plant by applying (for example by spraying an area) the molecules of Formula One to a different portion of the plant. For example, control of foliar-feeding insects can be achieved by drip irrigation or furrow application, by treating the soil with for example pre- or post-planting soil drench, or by treating the seeds of a plant before planting.

Seed treatment can be applied to all types of seeds, including those from which plants genetically modified to express specialized traits will germinate. Representative examples include those expressing proteins toxic to invertebrate pests, such as Bacillus thuringiensis or other insecticidal toxins, those expressing herbicide resistance, such as “Roundup Ready” seed, or those with “stacked” foreign genes expressing insecticidal toxins, herbicide resistance, nutrition-enhancement, drought resistance, or any other beneficial traits. Furthermore, such seed treatments with the molecules of Formula One may further enhance the ability of a plant to better withstand stressful growing conditions. This results in a healthier, more vigorous plant, which can lead to higher yields at harvest time. Generally, about 1 gram of the molecules of Formula One to about 500 grams per 100,000 seeds is expected to provide good benefits, amounts from about 10 grams to about 100 grams per 100,000 seeds is expected to provide better benefits, and amounts from about 25 grams to about 75 grams per 100,000 seeds is expected to provide even better benefits.

It should be readily apparent that the molecules of Formula One may be used on, in, or around plants genetically modified to express specialized traits, such as Bacillus thuringiensis or other insecticidal toxins, or those expressing herbicide resistance, or those with “stacked” foreign genes expressing insecticidal toxins, herbicide resistance, nutrition-enhancement, or any other beneficial traits.

The molecules of Formula One may be used for controlling endoparasites and ectoparasites in the veterinary medicine sector or in the field of non-human animal keeping. The molecules of Formula One are applied, such as by oral administration in the form of, for example, tablets, capsules, drinks, granules, by dermal application in the form of, for example, dipping, spraying, pouring on, spotting on, and dusting, and by parenteral administration in the form of, for example, an injection.

The molecules of Formula One may also be employed advantageously in livestock keeping, for example, cattle, sheep, pigs, chickens, and geese. They may also be employed advantageously in pets such as, horses, dogs, and cats. Particular pests to control would be fleas and ticks that are bothersome to such animals. Suitable formulations are administered orally to the animals with the drinking water or feed. The dosages and formulations that are suitable depend on the species.

The molecules of Formula One may also be used for controlling parasitic worms, especially of the intestine, in the animals listed above.

The molecules of Formula One may also be employed in therapeutic methods for human health care. Such methods include, but are limited to, oral administration in the form of, for example, tablets, capsules, drinks, granules, and by dermal application.

Pests around the world have been migrating to new environments (for such pest) and thereafter becoming a new invasive species in such new environment. The molecules of Formula One may also be used on such new invasive species to control them in such new environment.

The molecules of Formula One may also be used in an area where plants, such as crops, are growing (e.g. pre-planting, planting, pre-harvesting) and where there are low levels (even no actual presence) of pests that can commercially damage such plants. The use of such molecules in such area is to benefit the plants being grown in the area. Such benefits, may include, but are not limited to, improving the health of a plant, improving the yield of a plant (e.g. increased biomass and/or increased content of valuable ingredients), improving the vigor of a plant (e.g. improved plant growth and/or greener leaves), improving the quality of a plant (e.g. improved content or composition of certain ingredients), and improving the tolerance to abiotic and/or biotic stress of the plant.

Before a pesticide can be used or sold commercially, such pesticide undergoes lengthy evaluation processes by various governmental authorities (local, regional, state, national, and international). Voluminous data requirements are specified by regulatory authorities and must be addressed through data generation and submission by the product registrant or by a third party on the product registrant's behalf, often using a computer with a connection to the World Wide Web. These governmental authorities then review such data and if a determination of safety is concluded, provide the potential user or seller with product registration approval. Thereafter, in that locality where the product registration is granted and supported, such user or seller may use or sell such pesticide.

A molecule according to Formula One can be tested to determine its efficacy against pests. Furthermore, mode of action studies can be conducted to determine if said molecule has a different mode of action than other pesticides. Thereafter, such acquired data can be disseminated, such as by the internet, to third parties.

The headings in this document are for convenience only and must not be used to interpret any portion hereof.

### Table Section

TABLE 1
Compound number and structure

| Compound | |
|---|---|
| No. | Structure |
| 1 | 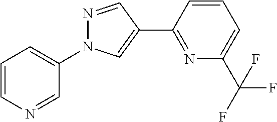 |
| 2 | 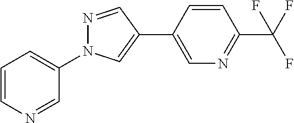 |
| 3 | 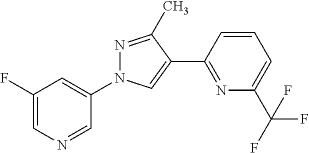 |
| 4 | 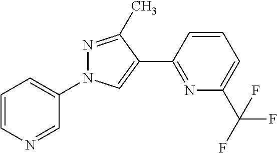 |
| 5 | 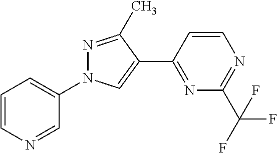 |
| 7 | 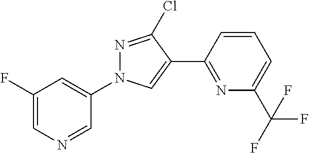 |
| 8 | 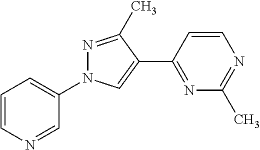 |
| 9 | 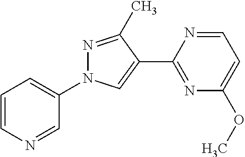 |
| 10 | 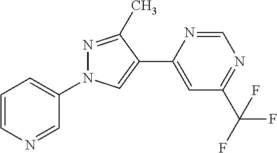 |
| 11 | 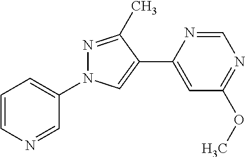 |
| 12 | 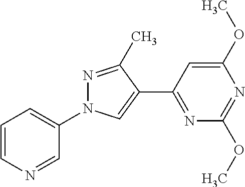 |
| 13 | 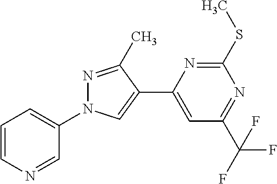 |
| 17 | 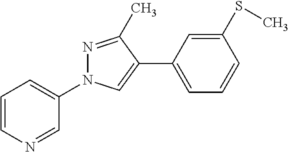 |
| 19 | 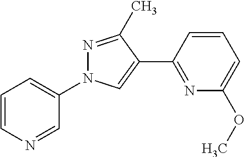 |
| 21 | 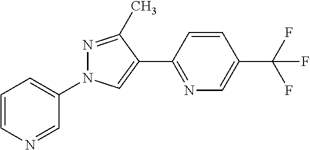 |
| 22 | 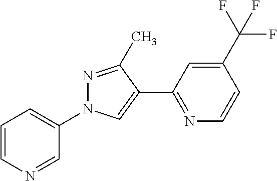 |
| 23 | 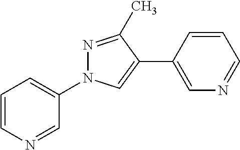 |
| 25 | 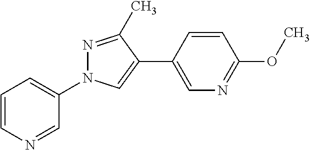 |
| 26 | 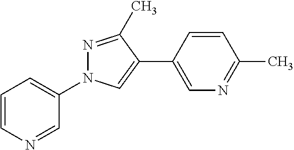 |
| 27 | 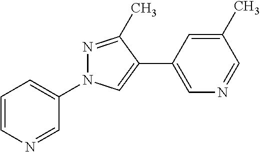 |
| 28 | 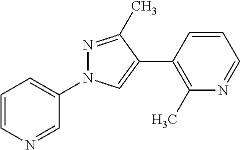 |
| 29 | 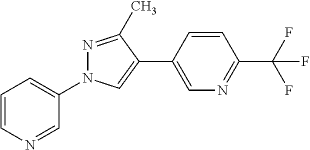 |
| 30 | 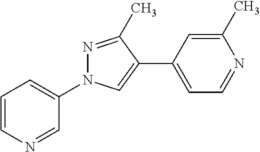 |

TABLE 2
Compound number and analytical data

| NO. | APPEARANCE | MP (° C.) | MASS | NMR |
|---|---|---|---|---|
| 1 | tan solid | 130-131 | EIMS m/z 290 | ¹H NMR (400 MHz, acetone-d₆) δ 9.24 |
| | | | | (d, J = 2.4 Hz, 1H), 9.13 (d, J = 0.5 Hz, |
| | | | | 1H), 8.60 (dd, J = 4.7, 1.4 Hz, 1H), 8.44 |
| | | | | (s, 1H), 8.35 (ddd, J = 8.3, 2.7, 1.5 Hz, |
| | | | | 1H), 8.12 (qd, J = 8.3, 4.7 Hz, 2H), 7.72 |
| | | | | (dd, J = 7.1, 1.5 Hz, 1H), 7.59 (ddd, J = |
| | | | | 8.3, 4.7, 0.7 Hz, 1H) |
| 2 | white solid | 203-204 | EIMS m/z 290 | ¹H NMR (400 MHz, Acetone) δ 9.21 (d, |
| | | | | J = 2.3 Hz, 1H), 9.18 (dd, J = 2.9, 1.4 |
| | | | | Hz, 2H), 8.61 (dd, J = 4.7, 1.4 Hz, 1H), |
| | | | | 8.44 (d, J = 0.4 Hz, 1H), 8.42-8.37 (m, |
| | | | | 1H), 8.32 (ddd, J = 8.3, 2.7, 1.5 Hz, 1H), |
| | | | | 7.91 (dd, J = 8.2, 0.6 Hz, 1H), 7.60 (ddd, |
| | | | | J = 8.3, 4.7, 0.7 Hz, 1H) |
| 3 | White Solid | 130-131 | EIMS m/z 322 | ¹H NMR (400 MHz, acetone-d₆ ) δ 9.15- |
| | | | | 9.09 (m, 1H), 9.09-9.04 (m, 1H), |
| | | | | 8.50-8.44 (m, 1H), 8.16-8.09 (m, |
| | | | | 2H), 8.03 (d, J = 7.8 Hz, 1H), 7.71 (dd, J = |
| | | | | 7.6, 0.8 Hz, 1H), 2.73-2.62 (m, 3H). |
| 4 | Tan Solid | 148-149 | EIMS m/z 304 | ¹H NMR (400 MHz, acetone-d₆) δ 9.18 |
| | | | | (d, J = 2.3 Hz, 1H), 9.05 (s, 1H), 8.54 (d, |
| | | | | J = 3.9 Hz, 1H), 8.27 (ddd, J = 8.3, 2.7, |
| | | | | 1.5 Hz, 1H), 8.15-8.00 (m, 2H), 7.69 |
| | | | | (dd, J = 7.6, 0.9 Hz, 1H), 7.54 (ddd, J = |
| | | | | 8.3, 4.7, 0.6 Hz, 1H), 2.67 (s, 3H). |
| 5 | Tan Solid | 170-171 | EIMS m/z 305 | ¹H NMR (400 MHz, acetone-d₆) δ 9.28 |
| | | | | (s, 1H), 9.18 (d, J = 2.5 Hz, 1H), 8.94 (d, |
| | | | | J = 5.4 Hz, 1H), 8.58 (dd, J = 4.7, 1.4 |
| | | | | Hz, 1H), 8.29 (ddd, J = 8.3, 2.7, 1.5 Hz, |
| | | | | 1H), 8.04 (d, J = 5.4 Hz, 1H), 7.56 (ddd, |
| | | | | J = 8.3, 4.7, 0.7 Hz, 1H), 2.71 (s, 3H). |
| 7 | White Solid | 116 | EIMS m/z 342 | ¹H NMR (400 MHz, acetone-d₆) δ 9.19 |
| | | | | (s, 1H), 9.15 - 9.10 (m, 1H), 8.58 (dd, J = |
| | | | | 2.5, 0.4 Hz, 1H), 8.32-8.19 (m, 3H), |
| | | | | 7.83 (dd, J = 7.4, 1.2 Hz, 1H). |
| 8 | White Solid | 174 | EIMS m/z 251 | ¹H NMR (400 MHz, acetone-d₆) δ 9.19- |
| | | | | 9.13 (m, 1H), 9.08 (s, 1H), 8.61 (d, J = |
| | | | | 5.3 Hz, 1H), 8.57-8.51 (m, 1H), 8.27 |
| | | | | (ddd, J = 8.3, 2.7, 1.4 Hz, 1H), 7.58- |
| | | | | 7.51 (m, 2H), 2.67 (s, 3H), 2.65-2.64 |
| | | | | (m, 3H). |
| 9 | White Solid | 103-104 | EIMS m/z 267 | ¹H NMR (400 MHz, acetone-d₆) δ 9.21 |
| | | | | (m, 1H), 9.01 (m, 1H), 8.56 (dd, J = 4.7, |
| | | | | 1.4 Hz, 1H), 8.51 (d, J = 5.8 Hz, 1H), |
| | | | | 8.31 (ddd, J = 8.3, 2.7, 1.4 Hz, 1H), 7.55 |
| | | | | (ddd, J = 8.3, 4.7, 0.8 Hz, 1H), 6.67 (d, J = |
| | | | | 5.8 Hz, 1H), 4.07 (s, 3H), 2.72 (m, |
| | | | | 3H). |
| 10 | Tan Solid | 137-138 | EIMS m/z 305 | ¹H NMR (400 MHz, acetone-d₆) δ 9.43- |
| | | | | 9.38 (m, 1H), 9.34 (d, J = 0.7 Hz, 1H), |
| | | | | 9.22-9.17 (m, 1H), 8.59 (dd, J = 4.7, |
| | | | | 1.4 Hz, 1H), 8.30 (ddd, J = 8.3, 2.7, 1.5 |
| | | | | Hz, 1H), 8.23 (d, J = 1.2 Hz, 1H), 7.58 |
| | | | | (ddd, J = 8.3, 4.7, 0.7 Hz, 1H), 2.73 (s, |
| | | | | 3H). |
| 11 | Yellow Solid | 188 | EIMS m/z 267 | ¹H NMR (400 MHz, DMSO-d₆) δ 9.29 |
| | | | | (s, 1H), 9.13 (d, J = 2.2 Hz, 1H), 8.81 (d, |
| | | | | J = 1.1 Hz, 1H), 8.55 (dd, J = 4.7, 1.4 |
| | | | | Hz, 1H), 8.26 (ddd, J = 8.4, 2.7, 1.4 Hz, |
| | | | | 1H), 7.57 (ddd, J =8.4, 4.7, 0.7 Hz, 1H), |
| | | | | 7.22 (d, J = 1.1 Hz, 1H), 3.97 (s, 3H), |
| | | | | 2.61 (s, 3H). |
| 12 | White Solid | 164-165 | EIMS m/z 297 | ¹H NMR (400 MHz, DMSO-d₆) δ 9.28 |
| | | | | (s, 1H), 9.12 (d, J = 2.3 Hz, 1H), 8.54 |
| | | | | (dd, J = 4.7, 1.4 Hz, 1H), 8.24 (ddd, J = |
| | | | | 8.4, 2.7, 1.4 Hz, 1H), 7.57 (ddd, J = 8.4, |
| | | | | 4.8, 0.6 Hz, 1H), 6.87 (s, 1H), 3.96 (s, |
| | | | | 3H), 3.91 (s, 3H), 2.62 (s, 3H). |
| 13 | Off White Solid | 183 | EIMS m/z 351 | ¹H NMR (400 MHz, acetone-d₆) δ 9.40 |
| | | | | (s, 1H), 9.20-9.15 (m, 1H), 8.58 (dd, J = |
| | | | | 4.7, 1.4 Hz, 1H), 8.28 (ddd, J = 8.3, |
| | | | | 2.7, 1.5 Hz, 1H), 7.86 (s, 1H), 7.56 (ddd, |
| | | | | J = 8.3, 4.7, 0.7 Hz, 1H), 2.72 (s, 1H), |
| | | | | 2.67 (s, 1H). |
| 17 | White Solid | 103.5-108.5 | EIMS m/z 281 | ¹H NMR (300 MHz, CDCl₃) δ 8.99 (d, J = |
| | | | | 2.5 Hz, 1H), 8.53 (dd, J = 4.7, 1.3 Hz, |
| | | | | 1H), 8.06 (ddd, J = 8.3, 2.6, 1.5 Hz, 1H), |
| | | | | 8.00 (s, 1H), 7.37 (m, 3H), 7.23 (m, 2H), |
| | | | | 2.53 (s, 3H), 2.49 (s, 3H). |
| 19 | Off White Solid | 108.6-112.6 | EIMS m/z 266 | ¹H NMR (400 MHz, CDCl₃) δ 9.02 (d, J = |
| | | | | 2.3 Hz, 1H), 8.54 (d, J = 3.7 Hz, 1H), |
| | | | | 8.34 (s, 1H), 8.08 (ddd, J = 8.3, 2.6, 1.5 |
| | | | | Hz, 1H), 7.60 (dd, J = 8.1, 7.5 Hz, 1H), |
| | | | | 7.41 (dd, J = 8.2, 4.7 Hz, 1H), 7.11 (d, J = |
| | | | | 7.3 Hz, 1H), 6.64 (d, J = 8.3 Hz, 1H), |
| | | | | 4.01 (s, 3H), 2.69 (s, 3H). |
| 21 | Off White Solid | 72.1-76.3 | ESIMS m/z | ¹H NMR (400 MHz, CDCl₃) δ 9.05 (s, |
| | | | 305([M + H]⁺) | 1H), 8.81 (d, J = 5.1 Hz, 1H), 8.57 (s, |
| | | | | 1H), 8.47 (s, 1H), 8.10 (d, J = 8.2 Hz, |
| | | | | 1H), 7.71 (s, 1H), 7.43 (dd, J = 8.2, 4.7 |
| | | | | Hz, 1H), 7.39 (d, J = 4.9 Hz, 1H), 2.67 |
| | | | | (s, 3H). |
| 22 | Brown Solid | 106.5-110.9 | EIMS m/z 304 | ¹H NMR (400 MHz, CDCl₃) δ 9.04 (s, |
| | | | | 1H), 8.58 (s, 1H), 8.47 (s, 1H), 8.09 (d, J = |
| | | | | 8.3 Hz, 1H), 7.85 (t, J = 7.9 Hz, 1H), |
| | | | | 7.73 (d, J = 8.1 Hz, 1H), 7.56 (d, J = 7.5 |
| | | | | Hz, 1H), 7.43 (dd, J = 8.0, 4.7 Hz, 1H), |
| | | | | 2.67 (s, 3H). |
| 23 | Pale Green Solid | 107.2-109.1 | EIMS m/z 236 | ¹H NMR (400 MHz, CDCl₃) δ 9.02 (s, |
| | | | | 1H), 8.80 (s, 1H), 8.59 (m, 2H), 8.07 (d, |
| | | | | J = 10.2 Hz, 2H), 7.77 (d, J = 7.8 Hz, |
| | | | | 1H), 7.42 (m, 2H), 2.51 (s, 3H). |
| 25 | Grey Solid | 103.1-105.3 | EIMS m/z 266 | ¹H NMR (400 MHz, CDCl₃) δ 8.99 (d, J = |
| | | | | 2.4 Hz, 1H), 8.54 (d, J = 4.0 Hz, 1H), |
| | | | | 8.27 (d, J = 2.3 Hz, 1H), 8.06 (d, J = 8.3 |
| | | | | Hz, 1H), 7.96 (s, 1H), 7.65 (dd, J = 8.5, |
| | | | | 2.4 Hz, 1H), 7.41 (dd, J = 8.3, 4.7 Hz, |
| | | | | 1H), 6.83 (d, J = 8.5 Hz, 1H), 3.98 (s, |
| | | | | 3H), 2.46 (s, 3H). |
| 26 | White Solid | 150.4-152.1 | EIMS m/z 250 | ¹H NMR (400 MHz, CDCl₃) δ 9.00 (d, J = |
| | | | | 2.3 Hz, 1H), 8.62 (s, 1H), 8.54 (d, J = |
| | | | | 4.6 Hz, 1H), 8.06 (d, J = 8.3 Hz, 1H), |
| | | | | 8.02 (s, 1H), 7.66 (dd, J = 8.0, 2.2 Hz, |
| | | | | 1H), 7.41 (dd, J = 8.3, 4.8 Hz, 1H), 7.23 |
| | | | | (d, J = 8.0 Hz, 1H), 2.61 (s, 3H), 2.48 (s, |
| | | | | 3H). |
| 27 | Grey Solid | 148.7-150.6 | EIMS m/z 250 | ¹H NMR (400 MHz, CDCl₃) δ 9.00 (d, J = |
| | | | | 2.4 Hz, 1H), 8.55 (d, J = 2.8 Hz, 2H), |
| | | | | 8.41 (s, 1H), 8.07 (d, J = 8.4 Hz, 1H), |
| | | | | 8.04 (s, 1H), 7.57 (s, 1H), 7.42 (dd, J = |
| | | | | 8.3, 4.7 Hz, 1H), 2.50 (s, 2H), 2.41 (s, |
| | | | | 3H). |
| 28 | White Solid | 113.1-117.3 | EIMS m/z 250 | ¹H NMR (400 MHz, CDCl₃) δ 8.99 (d, J = |
| | | | | 2.5 Hz, 1H), 8.54 (m, 2H), 8.07 (ddd, J = |
| | | | | 8.3, 2.6, 1.5 Hz, 1H), 7.89 (s, 1H), |
| | | | | 7.54 (dd, J = 7.6, 1.7 Hz, 1H), 7.42 (m, |
| | | | | 1H), 7.21 (dd, J = 7.6, 4.9 Hz, 1H), 2.53 |
| | | | | (s, 3H), 2.27 (s, 3H). |
| 29 | Grey Solid | 157.3-158.8 | EIMS m/z 304 | ¹H NMR (400 MHz, CDCl₃) δ 9.02 (d, J = |
| | | | | 2.2 Hz, 1H), 8.86 (s, 1H), 8.58 (d, J = |
| | | | | 4.5 Hz, 1H), 8.13 (s, 1H), 8.08 (d, J = |
| | | | | 8.3 Hz, 1H), 7.94 (d, J = 8.0 Hz, 1H), |
| | | | | 7.76 (d, J = 8.1 Hz, 1H), 7.44 (dd, J = |
| | | | | 8.2, 4.8 Hz, 1H), 2.53 (s, 3H). |
| 30 | Grey Solid | 130.9-133.2 | ESIMS m/z | ¹H NMR (400 MHz, CDCl₃) δ 9.00 (d, J = |
| | | | 251([M + H]⁺) | 2.5 Hz, 1H), 8.55 (dd, J = 12.9, 4.8 |
| | | | | Hz, 2H), 8.13 (s, 1H), 8.07 (d, J = 8.3 |
| | | | | Hz, 1H), 7.43 (dd, J = 8.3, 4.7 Hz, 1H), |
| | | | | 7.21 (d, J = 5.0 Hz, 1H), 2.62 (s, 3H), |
| | | | | 2.54 (s, 3H). |

TABLE 3
BAW & CEW Rating Table
BAW & CEW Rating Table

| % Control (or Mortality) | Rating |
|---|---|
| 50-100 | A |
| More than 0 - Less than 50 | B |
| Not Tested | C |
| No activity noticed in this bioassay | D |

TABLE 4
GPA, Scale, WF Rating Table

| % Control (or Mortality) | Rating |
|---|---|
| 80-100 | A |
| More than 0 - Less than 80 | B |
| Not Tested | C |
| No activity noticed in this bioassay | D |

TABLE 5
BAW, CEW, GPA, Scale and WF Results

| | BAW | CEW | GPA | Scale | WF |
|---|---|---|---|---|---|
| Com- | @ | @ | @ | @ | @ |
| pound | 200 | 200 | 200 | 200 | 200 |
| No. | ppm | ppm | ppm | ppm | ppm |
| 1 | A | B | A | A | B |
| 2 | A | D | A | D | C |
| 3 | A | D | A | A | C |
| 4 | A | C | A | A | C |
| 5 | A | C | A | A | C |
| 7 | A | D | A | A | C |
| 8 | A | C | A | A | C |
| 9 | A | C | A | A | C |
| 10 | A | C | A | A | C |
| 11 | A | C | A | B | C |
| 12 | A | C | A | B | C |
| 13 | A | C | B | B | C |
| 17 | A | C | A | B | D |
| 19 | A | C | A | A | B |
| 21 | A | C | A | A | A |
| 22 | A | C | A | A | A |
| 23 | A | C | A | A | B |
| 25 | A | C | A | A | B |
| 26 | A | C | A | A | A |
| 27 | A | C | A | B | A |
| 28 | A | C | A | A | B |
| 29 | A | C | A | A | A |
| 30 | A | C | A | B | B |

We claim:

1. A molecule according to Formula One: 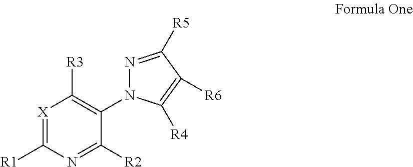
wherein:
(a) X is selected from N or CR12;
(b) R1 is selected from
(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));
(c) R2 is selected from
(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));
(d) R3 is selected from
(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));
(e) R4 is selected from
(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));
(f) R5 is selected from
(1) F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)), or
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13));
(g) R6 is selected from one of the following (6a), (6b), (6c), (6d), (6e), (6f), or (6g) 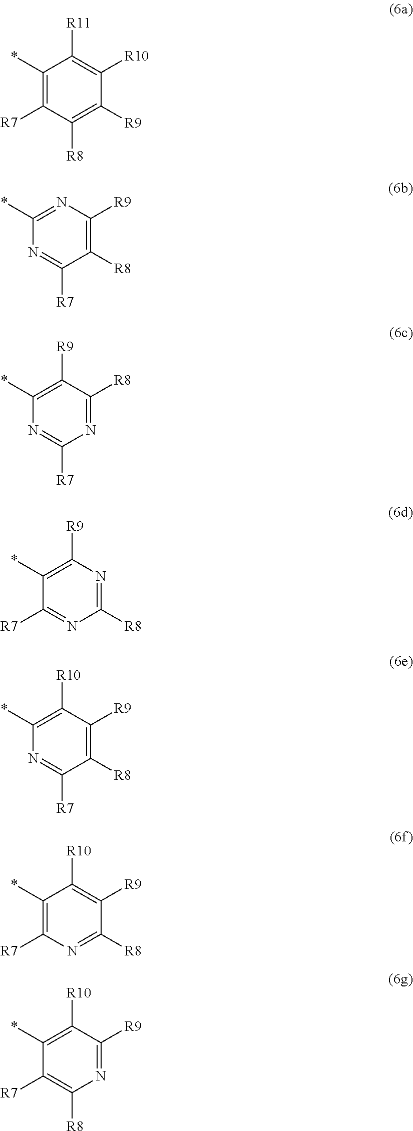
wherein * indicates the bond that is attached to the pyrazolyl ring;
(h) each R7 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(i) each R8 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(j) each R9 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(k) each R10 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(l) each R11 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(m) R12 is selected from
(1) H, F, Cl, Br, I, CN, NO₂, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, O(R13), C(═O)(R13), C(═S)(R13), C(═O)O(R13), C(═S)O(R13), C(═O)N(R13)₂, C(═S)N(R13)₂, N(R13)₂, N(R13)C(═O)(R13), N(R13)C(═S)(R13), S(R13), SO(R13), S(O)O(R13), S(O)₂O(R13), (R13)S(R13), (R13)S(O)(R13), (R13)S(O)₂(R13),
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13)),
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted, may optionally be substituted with (R13)), or
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(R13), S(O)ₙO(R13), (C₆-C₂₀)aryl, or (C₁-C₂₀)heterocyclyl, each of which that can be substituted may optionally be substituted with (R13));
(n) each R13 is independently selected from
(1) H, CN, (C₁-C₆)alkyl, (C₂-C₆)alkenyl, (C₁-C₆)alkoxy, (C₂-C₆)alkenyloxy, (C₃-C₁₀)cycloalkyl, (C₃-C₁₀)cycloalkenyl, (C₆-C₂₀)aryl, (C₁-C₂₀)heterocyclyl, S(C₁-C₆)alkyl, S(O)(C₁-C₆)alkyl, S(O)₂(C₁-C₆)alkyl, N((C₁-C₆)alkyl)₂,
(2) substituted (C₁-C₆)alkyl (wherein said substituted (C₁-C₆)alkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(3) substituted (C₂-C₆)alkenyl (wherein said substituted (C₂-C₆)alkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(4) substituted (C₁-C₆)alkoxy (wherein said substituted (C₁-C₆)alkoxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(5) substituted (C₂-C₆)alkenyloxy (wherein said substituted (C₂-C₆)alkenyloxy has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(6) substituted (C₃-C₁₀)cycloalkyl (wherein said substituted (C₃-C₁₀)cycloalkyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(7) substituted (C₃-C₁₀)cycloalkenyl (wherein said substituted (C₃-C₁₀)cycloalkenyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(8) substituted (C₆-C₂₀)aryl (wherein said substituted (C₆-C₂₀)aryl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)halo alkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl,
(9) substituted (C₁-C₂₀)heterocyclyl (wherein said substituted (C₁-C₂₀)heterocyclyl has one or more substituents selected from F, Cl, Br, I, CN, NO₂, (C₁-C₆)alkyl, (C₂-C₆)alkenyl, (C₁-C₆)haloalkyl, (C₂-C₆)haloalkenyl, (C₁-C₆)haloalkyloxy, (C₂-C₆)haloalkenyloxy, (C₃-C₁₀)cycloalkyl, (C₃-C₁₀)cycloalkenyl, (C₃-C₁₀)halocycloalkyl, (C₃-C₁₀)halocycloalkenyl, O(C₁-C₆)alkyl, O(C₁-C₆)haloalkyl, S(C₁-C₆)alkyl, S(O)(C₁-C₆)alkyl, S(O)₂(C₁-C₆)alkyl, SO(C₁-C₆)alkyl, S(O)O(C₁-C₆)alkyl, S(O)₂O(C₁-C₆)alkyl, (C₆-C₂₀)aryl, or (C₁-C₂₀)heterocyclyl;
(o) with the following provisos
(1) the following compounds are excluded

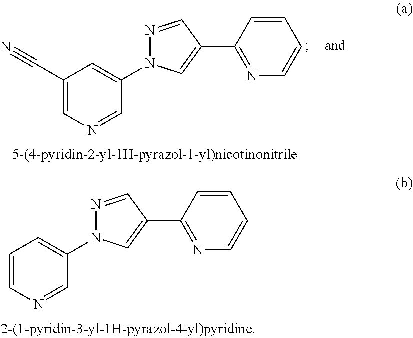

2. A molecule according to claim 1 having a structure according to one of the following compounds

| Compound | |
|---|---|
| No. | Structure |
| 3 | 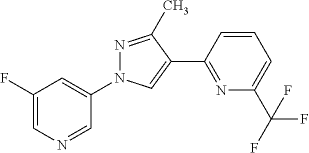 |
| 4 | 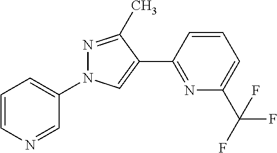 |
| 5 | 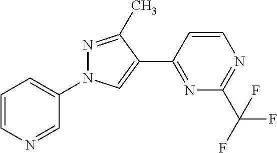 |
| 7 | 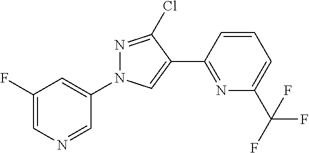 |
| 8 | 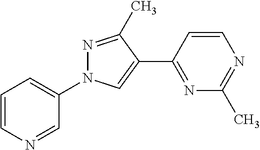 |
| 9 | 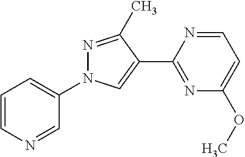 |
| 10 | 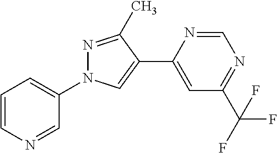 |
| 11 | 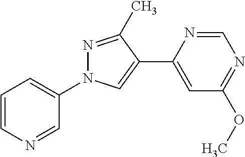 |
| 12 | 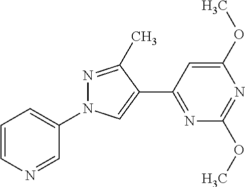 |
| 13 | 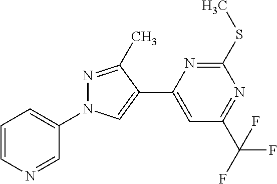 |
| 17 | 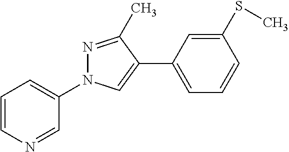 |
| 19 | 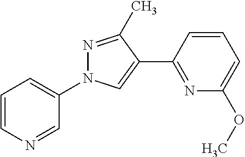 |
| 21 | 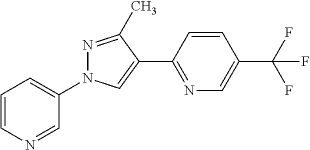 |
| 22 | 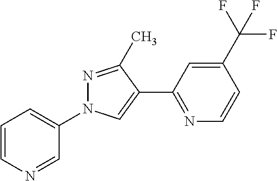 |
| 23 | 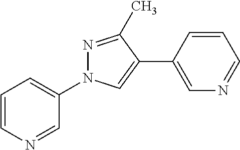 |
| 25 | 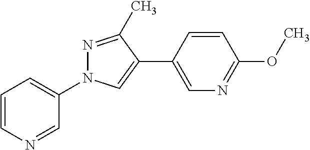 |
| 26 | 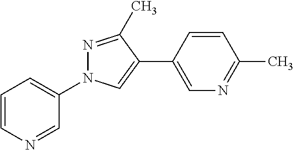 |
| 27 | 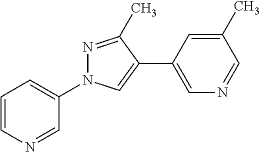 |
| 28 | 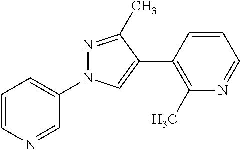 |
| 29 | 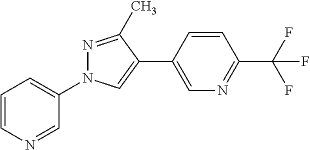 |
| 30 | 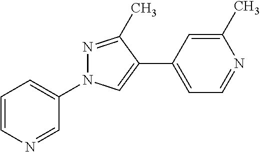 |

3. A process to apply a molecule according to claim 1 or 2 said process comprising applying a molecule according to claim 1 or 2, to an area to control a pest, in an amount sufficient to control such pest.

4. A process according to claim 3 wherein said pest is Beet Armyworm, Corn Earworm, Green Peach Aphid, Sweet Potato Whitefly, or San Jose Scale.

5. A process according to claim 3 wherein said area is an area where apples, corn, cotton, soybeans, canola, wheat, rice, sorghum, barley, oats, potatoes, oranges, alfalfa, lettuce, strawberries, tomatoes, peppers, crucifers, pears, tobacco, almonds, sugar beets, or beans, are growing, or the seeds thereof are going to be planted.

6. A molecule that is a pesticidally acceptable acid addition salt, a salt derivative, or a solvate, of a molecule according to claim 1 or 2.

7. A polymorph of a molecule according to claim 1 or 2.

8. A molecule according to claim 1 or 2 wherein at least one H is ²H or at least one C is ¹⁴C.

9. A composition comprising a molecule according to claim 1 or 2 and at least one other compound selected from the Insecticide Group, Acaricide Group, Nematicide Group, Fungicide Group, Herbicide Group, AI Group, or Synergist Group.

10. A composition comprising a molecule according to claim 1 or 2 and a seed.

11. A composition according to claim 10 wherein said seed has been genetically modified to express one or more specialized traits.

12. A composition comprising one molecule according to claim 1 or 2 and one molecule that has a mode of action selected from acetylcholinesterase inhibitor, sodium channel modulator, chitin biosynthesis inhibitor, GABA-gated chloride channel antagonist, GABA and glutamate-gated chloride channel agonist, acetylcholine receptor agonist, MET I inhibitor, Mg-stimulated ATPase inhibitor, nicotinic acetylcholine receptor, Midgut membrane disrupter, oxidative phosphorylation disrupter, and ryanodine receptor (RyRs).

13. A process comprising applying a molecule according to claim 1 or 2 to a genetically modified plant that has been genetically modified to express one or more specialized traits.

14. A process comprising: orally administering; or topically applying; a molecule according to claim 1 or 2, to a non-human animal, to control endoparasites, ectoparasites, or both.

15. A molecule according to claim 1, wherein
(a) X is CR12;
(b) R1 is H;
(c) R2 is H;
(d) R3 is H;
(e) R4 is H;
(f) R5 is selected from F, Cl, Br, I, or (C₁-C₆)alkyl;
(g) R6 is selected from one of the following (6a), (6b), (6c), (6d), (6e), (6f), or (6g) 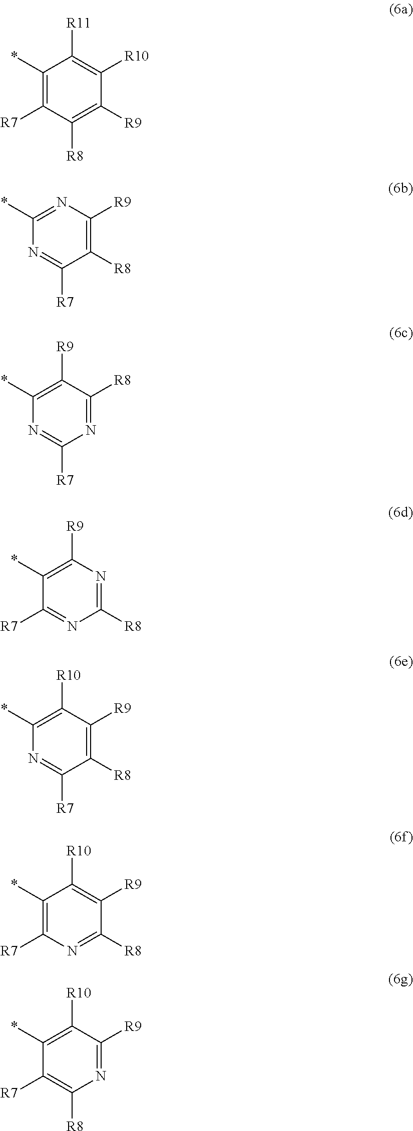
wherein * indicates the bond that is attached to the pyrazolyl ring;
(h) each R7 is independently selected from H, (C₁-C₆)alkyl, (C₁-C₆)alkoxy, S(C₁-C₆)alkyl, substituted (C₁-C₆)alkyl wherein said substituted (C₁-C₆)alkyl has one or more substituents selected from F, Cl, Br, I;
(i) each R8 is independently selected from H, (C₁-C₆)alkyl, (C₁-C₆)alkoxy, substituted (C₁-C₆)alkyl wherein said substituted (C₁-C₆)alkyl has one or more substituents selected from F, Cl, Br, I;
(j) each R9 is independently selected from H, (C₁-C₆)alkyl, substituted (C₁-C₆)alkyl wherein said substituted (C₁-C₆)alkyl has one or more substituents selected from F, Cl, Br, I;
(k) each R10 is H;
(l) each R11 is H;
(m) R12 is selected from H, F, Cl, Br, I.
